(19) United States
(12) Patent Application Publication
(10) Pub. No.: US 2017/0283854 A1
(43) Pub. Date: Oct. 5, 2017

(54) MULTIPLEXED PCR ASSAY FOR HIGH THROUGHPUT GENOTYPING

(71) Applicants: University of Florida Research Foundation, Inc., Gainesville, FL (US); Rapid Genomics, Gainesville, FL (US)

(72) Inventors: Christopher Dervinis, High Springs, FL (US); Matias Kirst, Gainesville, FL (US); Leandro G. Neves, Gainesville, FL (US); Marcio F. Resende, Gainesville, FL (US)

(21) Appl. No.: 15/505,568

(22) PCT Filed: Aug. 26, 2015
(86) PCT No.: PCT/US15/46899

§ 371 Date: Feb. 21, 2017

Related U.S. Application Data
(60) Provisional application No. 62/046,795, filed on Sep. 5, 2014.

(51) Int. Cl. C12Q 1/68; G06F 19/10; C40B 40/06

(52) U.S. Cl. CPC C12Q 1/68; C40B 40/06; C12Q 1/6869; G06F 19/10; C40B 40/00

(57) ABSTRACT

The present invention relates to identification and characterization of polymorphisms in a nucleic acid sample. Methods and compositions for the unbiased amplification of multiple target sequences within a nucleic acid sample are provided.

### REFERENCE TO RELATED APPLICATIONS

This application claims the benefit of U.S. provisional application No. 62/046,795, filed Sep. 5, 2014, and is herein incorporated by reference in its entirety.

### INCORPORATION OF SEQUENCE LISTING

The sequence listing that is contained in the file named “UFFL056WO_ST25.txt,” which is 54.1 kilobytes as measured in Microsoft Windows operating system and was created on Aug. 25, 2015, is filed electronically herewith and incorporated herein by reference.

### FIELD OF THE INVENTION

The present invention relates to the fields of molecular biology and genetics. In specific embodiments, the invention relates to identification and characterization of polymorphisms in a nucleic acid sample.

### INCORPORATION OF SEQUENCE LISTING

A sequence listing contained in the file named “UFFL056USP1_ST25.txt” which is 55 kilobytes (measured in MS-Windows®) and created on Sep. 5, 2014, and comprises 304 nucleotide sequences, is filed electronically herewith and incorporated by reference in its entirety.

### BACKGROUND OF THE INVENTION

The ability to select individuals for breeding based on a favorable genotype at a polymorphic locus is an important tool in plant and animal breeding technology. In addition, the ability to screen polymorphic markers for a parentage assay that is reliable and effective in any species depends on the accurate genotyping of a number of polymorphic loci. However, the use of polymorphic markers in breeding programs is greatly complicated by polygenic inheritance and epistasis, which can necessitate genotyping of a number of distinct polymorphic loci in an individual to gain useful information regarding a particular trait. The process of genotyping numerous polymorphic loci simultaneously is laborious and costly, and existing methods of doing so are frequently inaccurate. In the absence of new methods for efficiently and reliably detecting the genotype of an individual at a plurality of polymorphic genomic loci, the use of large numbers of polymorphic markers for selection in breeding programs may not be practical.

### SUMMARY OF THE INVENTION

In one aspect, the invention provides a method for genotyping, that may be implemented using next-generation sequencing, one or more target loci in a nucleic acid sample, comprising the steps of: a) providing a nucleic acid sample; b) adding a first set of primers to the sample to form a first amplification mixture, wherein the primers in the first set comprise a primer tail sequence and are capable of hybridizing to the target sequence within or adjacent to one or more of the target loci; c) performing a first amplification reaction on the first amplification mixture to produce a first library of amplicons, wherein the amplicons comprise the primer tail sequence; d) adding a second set of primers to the first library to form a second amplification mixture, wherein the primers in the second set are capable of hybridizing to the primer tail sequence; and e) performing a second amplification reaction on the second amplification mixture to generate a second library of amplicons; wherein for at least 90% of the target loci, the number of amplicons in the second library derived from each of the target loci deviates from the average number of amplicons for all target loci by less than one order of magnitude (+ or −10×).

In one embodiment of a method of the invention, for at least 90% of the target loci, the number of amplicons in the second library derived from each of the target loci deviates from the average number of amplicons for all target loci by less than 5× or less than 2.5×. In another embodiment, the number of target loci is greater than 10, greater than 100, or greater than 1,000. In a method of the invention, the first amplification reaction and the second amplification reaction can be carried out simultaneously or consecutively. In a specific embodiment, the first amplification reaction is carried out before the second amplification reaction. The method may comprise purifying the first library after the first amplification reaction and before the second amplification reaction. In certain further embodiment, the first and/or second amplification reaction comprises at least 2 cycles, at least 5 cycles, at least 10 cycles, at least 25 cycles, at least 50 cycles, between 5 and 50 cycles, between 1 and 15 cycles, between 2 and 10 cycles, or between 4 and 6 cycles. The primers in the first primer set are in one embodiment present in varying concentrations. The concentrations of the primers may be calculated according to a regression equation. In one embodiment, the target loci are polymorphic genomic loci within a population. In yet another embodiment, one or more primers used with the invention contain a unique index/barcode sequence to distinguish the sequencing of a sample or multiple samples in parallel.

In another aspect, a method of the invention further comprising the steps of: f) obtaining sequence data from the first or the second library; and h) determining the genotype at one or more of the target loci from the sequence data.

In yet another aspect, the invention provides a method for identifying a novel polymorphic genomic locus in a sample, comprising the steps of: a) providing two or more samples from individuals in a population; b) subjecting each of the samples to the method of claim 17; and c) aligning sequences corresponding to one or more target loci from two or more samples to identify target loci having sequence variation between individuals. Still further provided by the invention is a kit for use in genotyping one or more target loci in a nucleic acid sample, comprising: a) a first set of primers, wherein each primer in the first set comprises a primer tail sequence and is capable of hybridizing to a target sequence; and b) a second set of primers, wherein each primer in the second set is capable of hybridizing to the primer tail sequence.

### BRIEF DESCRIPTION OF THE DRAWINGS

FIG. 1 shows steps in a method of creating a library for detection of a target polymorphism according to the present invention. The target polymorphism and/or target region is shaded in grey. Primers (arrow) containing a tail (diagonal line) are used to amplify the target region or polymorphism. Following incorporation of the tail in the shortest possible number of cycles in a linear amplification, a second primer (arrow) containing a tail (diagonal line), is used to amplify the target region or polymorphism.

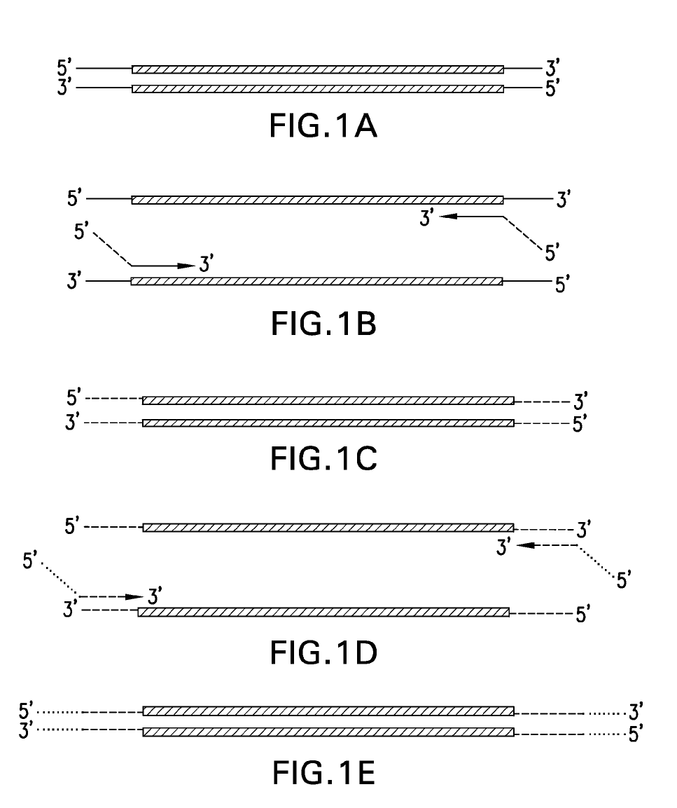

### DETAILED DESCRIPTION

Selection of individuals in a breeding population based on genetic markers linked to traits of interest is an essential tool in animal and plant breeding. Genetic markers can be used to accurately identify and track a desired trait within a breeding population, and allow for detection of a desired trait without the need to grow large populations to maturity in order to observe individual phenotypes. However, existing technology such as whole-genome sequencing or sequence-capture for the detection or discovery of polymorphic genetic markers is labor-intensive, expensive, and often inaccurate. In addition, the ability to screen polymorphic markers for a parentage assay that is reliable and effective in any species has been hampered by limitations on accurately genotyping multiple polymorphic loci.

While polymerase chain reaction (PCR)-based methods have been used for the detection of known genetic markers, these methods are typically not suitable for analysis of large numbers of polymorphic markers in a sample, such as with implementation of next generation sequencing methods. This is due in part to the difficulty in producing a consistent PCR amplification of a large number of target sequences in a single sample. This difficulty arises from variation in the efficiency by which distinct target sequences are amplified, due in part to differences in primer hybridization kinetics between target sequences. Small differences in primer annealing properties result in a biased amplification that is propagated exponentially with additional amplification cycles. Although amplification bias can be minimized by reducing the number of cycles in an amplification reaction, this results in insufficient amplified product for subsequent analysis of genetic markers. On the other hand, if a large number of cycles are carried out, amplification bias results in the detection of high numbers of amplicons corresponding to one or a few loci, while other loci are under-represented or not detected at all.

The present invention solves this problem by providing methods for amplifying a large number of loci from genomic DNA in an unbiased manner. In some embodiments, the methods of the invention comprise a two-step amplification reaction. In the first amplification step, locus specific primers comprising a primer tail are used for amplification for only few cycles to prevent the development of significant amplification bias. In the second amplification step, universal primers specific to the primer tail introduced in the first amplification step are used for further amplification in an unbiased manner. Using this approach, the final number of reads obtained by sequencing of the amplification products is consistent across loci.

Embodiments of the present invention therefore advantageously provide methods for unbiased amplification of sequences from multiple loci within a sample. For example, the number of loci to be detected in a sample may be 10 or more, 100 or more, or 1,000 or more loci, including, for example, from a lower range of about 5, 10, 25, 50, 75, 100, 150, 200, 250 or 500 or more to about 50, 75, 100, 150, 200, 300, 400, 500, 750, 1,000, or 1500 or more, including all combinations thereof. In further embodiments, the invention provides methods for amplifying multiple loci within a sample such that the final number of amplicons derived from each locus is balanced across loci. In particular embodiments, the invention provides methods for amplifying multiple loci within a sample such that for at least 90% of the loci tested, the final number of amplicons derived from any particular locus deviates from the average number of amplicons for all loci by one order of magnitude (i.e. + or −10×). For instance, if on average the number of reads obtained for all amplicons is 1% of the total number of reads, then an expected maximum of 10% and a minimum of 0.1% of reads would be detected for at least 90% of the other loci. In other embodiments, the method for amplifying multiple loci within a sample is such that for at least 80%, 85%, 90% or 95% of the loci tested, the final number of amplicons derived from any particular locus deviates from the average number of amplicons for all loci by less than about 7.5×, 5×, 2.5×, 1.5×, 1×, or less than 0.5×.

The unbiased two-step amplification methods provided by the present invention may comprise a first amplification step which is carried out using the lowest number of cycles required to effectively create a first library of amplicons comprising primer tails. In certain embodiments, the first amplification reaction is carried out using between 1-15 cycles, 2-10 cycles, between 3-7 cycles, or between 4-6 cycles of amplification. In certain embodiments, the primers used in the first amplification step may be present in varying concentrations according to the specific loci to which they correspond. In other embodiments, the primers used in the first amplification step may be calculated or adjusted according to the following equation:

$${log}_{10} ( \frac{{reads}_{i} 100}{{\sum }_{i} {reads}_{i}} ) = {β}_{0} + {β}_{1} * {log}_{10} ( {dilution}_{i} )$$

Wherein reads stands for the number of reads sequenced for primer i and dilution stands for the concentration level used in the experiment for the same primer i. The regression coefficients β0 and β1 are calculated based on the data as described in Example 1, and for the test set in the example consisted of 0.34678 and 1.42626, respectively. These values can change for other test sets. The methods of the invention may further provide a second amplification step which amplifies a first library of amplicons by using primers directed to a primer tail which was added to the amplicons in the first amplification step. In certain embodiments, the second amplification reaction can comprise at least one 1 cycle, between 5 and 50 cycles, or between 10 and 25 cycles.

In specific embodiments, the first and the second amplification steps may be carried out simultaneously or consecutively. For example, the first amplification step may be carried out using a first set of primers at a concentration such that the amount of primer remaining after the first amplification step will be negligible. The second amplification step can be carried out consecutively or sequentially after the addition of a second primer set without the need for removing residual first primer. In other embodiments, the first and second amplification reactions may be carried out consecutively, and residual primer may be removed after the first purification step is complete. For example, a purification step may be used between the first amplification reaction and the second amplification reaction. A purification step may include any means known in the art for separating amplicons from a reaction mixture. In yet further embodiments, the first primer set and the second primer set are designed such that they hybridize with their specific target sequences under different conditions. The first and second amplification steps can then be carried out using differing temperature cycling protocols without the need for removal of residual primer between the steps.

The invention further provides methods for obtaining sequence data at one or more loci in a nucleic acid sample. For example, sequence data can be obtained from a first or second library produced by the first or the second amplification step by methods known in the art. The invention further contemplates determining the genotype at one or more target loci, for example one or more polymorphic genetic loci within a genomic DNA sample, from the sequence data. Further envisaged applications of the methods of the present invention include screening enriched microsatellite libraries, performing transcript profiling cDNA-AFLP (digital Northern), sequencing of complex genomes, EST library sequencing (on whole cDNA or cDNA-AFLP), microRNA discovery (sequencing of small insert libraries), bacterial artificial chromosome (BAC) contig sequencing, bulked segregant analysis approach AFLP/cDNA-AFLP, and detection of AFLP fragments, e.g. for marker-assisted selection (MAS) or marker-assisted back-crossing (MABC).

The invention further provides for genotyping a sample at one or more known polymorphic loci using the unbiased amplification methods provided herein. In other embodiments, the methods provide for identification of new polymorphic genomic loci within a population. In an exemplary embodiment, two or more samples are obtained from individuals in a population, and each of the samples is processed according to the methods of the present invention to provide sequence information for one or more target loci within the samples. Sequence data from the one or more individuals is then aligned to detect variations in sequences between individuals in the population, and variations in sequence within the population are used as genetic markers for tracking or identifying traits.

In yet a further embodiment, the invention provides kits for use in genotyping one or more target loci in a nucleic acid sample using the unbiased amplification methods provided herein. An exemplary kit according to the present invention comprises a first set of primers wherein each primer in the first set comprises a primer tail sequence and is capable of hybridizing to a target sequence, and a second set of primers wherein each primer in the second set is capable of hybridizing to the primer tail sequence. The kits provided by the invention may further provide reagents for carrying out nucleic acid amplification reactions, such as DNA polymerase, dideoxyribonucleotides with or without detectable labels, and buffer solutions. The kits of the invention may further provide instructions for using the kit components according to the methods provided herein.

### I. Molecular Markers

“Marker,” “genetic marker,” “molecular marker,” “marker nucleic acid,” and “marker locus” refer to a nucleotide sequence or encoded product thereof (e.g., a protein) used as a point of reference when identifying a DNA locus influencing a phenotype in an organism. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a quantitative trait locus (QTL), that are genetically or physically linked to the marker locus. Thus, a “marker allele,” alternatively an “allele of a marker locus,” is one of a plurality of nucleotide sequences found at a polymorphic marker locus in a population.

Markers that can be used in the practice of the present invention include, but are not limited to, unique expressed sequence tags (EST); restriction fragment length polymorphisms (RFLP), amplified fragment length polymorphisms (AFLP), simple sequence repeats (SSR), simple sequence length polymorphisms (SSLPs), single nucleotide polymorphisms (SNP), insertion/deletion polymorphisms (Indels), variable number tandem repeats (VNTR), and random amplified polymorphic DNA (RAPD), isozymes, and others known to those skilled in the art. Polymorphisms comprising as little as a single nucleotide change can be assayed in a number of ways. For example, detection can be made by electrophoretic techniques including a single strand conformational polymorphism (Orita et al. (1989) Genomics 8(2), 271-278), denaturing gradient gel electrophoresis (Myers (1985) EPO 0273085), or cleavage fragment length polymorphisms (Life Technologies, Inc., Gaithersburg, Md. 20877), or direct sequencing.

Once a polymorphism in a population is known, assays can be designed to detect alleles at the polymorphic locus in members of the population. Methods for detecting alleles at a polymorphic locus include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Methods are also known for the detection of expressed sequence tags (ESTs) and SSR markers derived from EST sequences and randomly amplified polymorphic DNA (RAPD).

A marker sequence typically comprises two alleles at each polymorphic locus in a diploid organism. A diploid individual can therefore be either homozygous or heterozygous at a given locus. Homozygosity is a condition in which both alleles at a locus are characterized by the same nucleotide sequence. Heterozygosity refers to the presence of two different alleles at a given locus in a diploid organism.

A favorable allele of a marker is the allele of the marker that co-segregates with a desired phenotype. As used herein, a marker has a minimum of one favorable allele, although it is possible that the marker might have two or more favorable alleles found in the population. Any favorable allele of that marker can be used advantageously for the identification and tracking of favorable traits in a breeding program. Alternatively, a marker allele that co-segregates with an undesirable phenotype may be useful in the invention, since that allele can be used to identify and counter select an unfavorable genotype. Such an allele can be used for exclusionary purposes during breeding to identify individuals having genotypes that negatively correlate with a desired phenotype for elimination during subsequent rounds of breeding.

The more tightly linked a marker is with a polymorphic locus influencing a phenotype, the more reliable the marker is in marker-assisted selection (MAS), as the likelihood of a recombination event unlinking the marker and the locus decreases. Markers containing the causal mutation for a trait, or that are within the coding sequence of a causative gene, are ideal as no recombination is expected between them and the sequence of DNA responsible for the phenotype.

Genetic markers are distinguishable from one another (as well as from the plurality of alleles of any one particular marker) on the basis of polynucleotide length and/or sequence. Genetic markers are known in the art for many well-characterized organisms, and novel markers may also be developed by methods known in the art. In general, any differentially inherited polymorphic trait (including a nucleic acid polymorphism) that segregates among progeny is a potential genetic marker.

### II. Marker Detection

Methods for determining the genotype of an organism at a given marker locus include, but are not limited to, PCR-based detection methods, microarray methods, mass spectrometry-based methods and nucleic acid sequencing methods, including whole genome sequencing. In certain embodiments, the detection of alleles at polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means. One method of achieving such amplification employs the polymerase chain reaction (PCR) (Mullis et al., 1986 Cold Spring Harbor Symp Quant Biol 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

In some aspects, methods of the invention utilize an amplification step to genotype a marker locus. Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

“Amplifying,” in the context of nucleic acid amplification, is any process whereby additional copies of a selected nucleic acid (or a transcribed form thereof) are produced. In some embodiments, an amplification-based marker technology is used wherein a primer or amplification primer pair is admixed with a nucleic acid sample from an organism, and wherein the primer or primer pair is complementary to or partially complementary to at least a portion of a marker locus, and is capable of initiating DNA polymerization by a DNA polymerase using the nucleic acid sample as a template. The primer or primer pair is extended in a DNA polymerization reaction having a DNA polymerase and a template genomic nucleic acid to generate at least one amplicon.

Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like). A “template nucleic acid” is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods are known in the art, and one of skill will appreciate that essentially any RNA can be converted into a double stranded DNA suitable for restriction digestion, PCR expansion, and sequencing using reverse transcriptase and a polymerase.

In some embodiments, the presence or absence of a molecular marker is determined through detection of a nucleic acid sequence at a polymorphic marker region. In alternative embodiments, in silico methods can be used to detect the marker loci of interest. For example, the sequence of a nucleic acid comprising a marker locus of interest can be stored in a computer. The desired marker locus sequence or its homolog can be identified using an appropriate nucleic acid search algorithm as provided by, for example, in such readily available programs as BLAST, or even simple word processors.

### III. Nucleic Acids

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. Polynucleotide sequences include the DNA strand sequence that is transcribed into RNA and the strand sequence that is complementary to the DNA strand that is transcribed. Polynucleotide sequences also include both full-length sequences as well as shorter sequences derived from the full-length sequences. Allelic variations of the exemplified sequences also fall within the scope of the subject invention. Polynucleotide sequences include both the sense and antisense strands either as individual strands or in the duplex. The nomenclature used herein is that required by Title 37 of the United States Code of Federal Regulations §1.822 and set forth in the tables in WIPO Standard ST.25 (1998), Appendix 2, Tables 1 and 3.

As used herein, the term “recombinant nucleic acid,” “recombinant polynucleotide” or “recombinant DNA molecule” refers to a polynucleotide that has been altered from its native state, such as by linkage to one or more other polynucleotide sequences to which the recombinant polynucleotide molecule is not normally linked to in nature. Such molecules may or may not be present, for example, in a host genome or chromosome.

The subject invention also concerns oligonucleotide probes and primers, such as polymerase chain reaction (PCR) primers, that can hybridize to a coding or non-coding sequence of a polynucleotide of the present invention. Oligonucleotide probes of the invention can be used in methods for detecting and quantitating nucleic acid sequences. Oligonucleotide primers of the invention can be used in PCR methods and other methods involving nucleic acid amplification. In a preferred embodiment, a probe or primer of the invention can hybridize to a polynucleotide of the invention under stringent conditions. Probes and primers of the invention can optionally comprise a detectable label or reporter molecule, such as fluorescent molecules, enzymes, radioactive moiety (e.g., ³H, ³⁵S, ¹²⁵I, etc.), and the like. Probes and primers of the invention can be of any suitable length for the method or assay in which they are being employed. Typically, probes and primers of the invention will be 10 to 500 or more nucleotides in length. Probes and primers of the invention can have complete (100%) nucleotide sequence identity with the polynucleotide sequence, or the sequence identity can be less than 100%. For example, sequence identity between a probe or primer and a sequence can be 70% or greater, 75% or greater, 80% or greater, 85% or greater, 90% or greater, or 95% to 100%, or any other percentage sequence identity allowing the probe or primer to hybridize under stringent conditions to a nucleotide sequence of a polynucleotide of the invention. In one embodiment, a probe or primer of the invention has 70% or greater, 75% or greater, 80% or greater, 85% or greater, 90% or greater, or 95% to 100% sequence identity with a nucleotide sequence provided herein, including the complement thereof.

### IV. Methods of Modifying Nucleic Acids

The subject invention also concerns variants of the polynucleotides of the present invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). Polynucleotides contemplated within the scope of the subject invention can also be defined in terms of identity and/or similarity ranges with those sequences of the invention specifically exemplified herein. In certain embodiments, the invention provides polynucleotide sequences having at least about 70, 80, 85, 90, 95, 99, or 99.5 percent identity to a polynucleotide sequence provided herein.

The invention also contemplates polynucleotide molecules having sequences which are sufficiently homologous with the polynucleotide sequences exemplified herein so as to permit hybridization with that sequence under standard stringent conditions and standard methods (Maniatis, et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature, Tₘ, is described by the following formula (Beltz, et al., 1983):

Tₘ=81.5C+16.6 Log [Na⁺]+0.41(% G+C)−0.61(% formamide)−600/length of duplex in base pairs.

Washes are typically carried out as follows:
(1) Twice at room temperature for 15 minutes in 1×SSPE, 0.1% SDS (low stringency wash).
(2) Once at Tₘ−20 C for 15 minutes in 0.2×SSPE, 0.1% SDS (moderate stringency wash).

### V. Probes and Primers

In general, synthetic methods for making oligonucleotides, including probes and primers, are known in the art. For example, oligonucleotides can be synthesized chemically according to the solid phase phosphoramidite triester method. Oligonucleotides, including modified oligonucleotides, can also be ordered from a variety of commercial sources.

Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the invention be limited to any particular size.

In some embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. As used in the art, PCR primers used to amplify a molecular marker are sometimes termed “PCR markers” or simply “markers.” It will be appreciated that, although specific examples of primers are provided herein, suitable primers to be used with the invention can be designed using any suitable method. It is not intended that the invention be limited to any particular primer or primer pair.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

### VI. Marker Assisted Selection

Marker discovery and development provides the initial framework for marker-assisted breeding programs. Marker-assisted selection (MAS) refers to the selection of individuals based on genetic markers linked to traits of interest during breeding. Individuals may be selected according to their genotype at one or a plurality of marker loci in MAS breeding programs.

In some embodiments of the invention, one or more marker alleles are selected for in a single organism or in a population. In these methods, individuals are selected that contain favorable alleles from more than one marker, or alternatively, favorable alleles from more than one marker are introgressed into a desired population. The determination of which marker alleles correlate with a favorable phenotype is determined for the particular germplasm under study. One of skill recognizes that methods for identifying the favorable alleles are routine and well known in the art, and furthermore, that the identification and use of such favorable alleles is well within the scope of this invention.

Methods of the present invention may evaluate traits including, but not limited to, complex/quantitative traits, monogenic traits, and/or polygenic traits. Such traits in plants may include, for example, reproductive health, plant height, yield, biomass, increased or decreased tolerance to stress, both biotic or abiotic, or to a chemical such as a pesticide or a herbicide, and the like. Such traits in animals may include, for example, weight, weaning weight, carcass composition such as marbling and back fat, hip structure, litter size, fertility, reproductive health, and the like. An “individual” or “subject” in accordance with the present invention may be a plant including, but not limited to an agricultural plant or tree. Agricultural plants or trees as used herein generally refer to plants and trees grown primarily for food or production purposes. Such plants and trees include but are not limited to rice, soybean, corn, canola, sorghum, sugarcane, cotton, coffee, tomato, pine, oak, maple, citrus, or the like. In addition, an “individual” or “subject” may be an animal including, but not limited to a livestock animal. Livestock animals as used herein generally refer to animals raised primarily for food. Such animals include, but are not limited to cattle, swine, horse, goat, sheep, dog, ostrich, chicken, turkey, and the like. As used herein, the term “plant” includes plant cells, plant protoplasts, plant cells of tissue culture from which plants can be regenerated, plant calli, plant clumps and plant cells that are intact in plants or parts of plants such as pollen, flowers, seeds, leaves, stems, and the like.

Certain embodiments of the invention provide early selection of an individual for breeding. Early selection may include selection of an individual for breeding before the individual fully exhibits a trait or phenotype, or before a trait is fully established in an individual.

Embodiments of the invention may provide a kit for determining the genotype of an individual. Such a kit may include means for detecting one or a plurality of genetic markers. In vitro test kits (e.g., reagent kits) for determining the genotype of an individual may include reagents, materials, and protocols for assessing one or more biomarkers (e.g., nucleic acids, proteins, or the like), instructions and, optionally, software for comparing the biomarker data between individuals. Useful reagents and materials for kits include, but are not limited to PCR primers, hybridization probes and primers (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, microarrays, antibodies, means for amplification of nucleic acid fragments from one or more individuals, means for analyzing the nucleic acid sequence of one or more individuals or fragments thereof, or means for analyzing the sequence of one or more amino acid residues from one or more individuals to be selected for breeding.

### VII. Definitions

The definitions and methods provided define the present invention and guide those of ordinary skill in the art in the practice of the present invention. Unless otherwise noted, terms are to be understood according to conventional usage by those of ordinary skill in the relevant art. Examples of resources describing many of the terms related to molecular biology used herein can be found in Alberts, et al., Molecular Biology of The Cell, 5th Edition, Garland Science Publishing, Inc.: New York, 2007; Rieger, et al., Glossary of Genetics: Classical and Molecular, 5th edition, Springer-Verlag: New York, 1991; King, et al., A Dictionary of Genetics, 6th ed, Oxford University Press: New York, 2002; and Lewin, Genes Icorn, Oxford University Press: New York, 2007. The nomenclature for DNA bases as set forth at 37 CFR §1.822 is used.

“Adjacent,” when used to describe a nucleic acid molecule that hybridizes to DNA containing a polymorphism, refers to DNA sequences that directly abut the polymorphic nucleotide base position. For example, a nucleic acid molecule that can be used in a single base extension assay is “adjacent” to the polymorphism.

“Allele” refers to an alternative nucleic acid sequence at a particular locus; the length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population. “Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to that trait and when presence of the allele is an indictor that the trait will occur in an individual. An allele negatively correlates with a trait when it is linked to the trait and when presence of the allele is an indicator that the trait will not occur in an individual.

“Genetic element” or “gene” refers to a heritable sequence of DNA, i.e., a genomic sequence, with functional significance. The term “gene” can also be used to refer to, e.g., a cDNA and/or an mRNA encoded by a genomic sequence, as well as to that genomic sequence.

“Genotype” is the genetic constitution of an individual (or group of individuals) at one or more genetic loci, as contrasted with the observable trait (the phenotype). Genotype is defined by the allele(s) at one or more loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome interval. The terms “phenotype,” or “phenotypic trait” or “trait” refers to one or more traits of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is controlled by a plurality of genes or genetic loci.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells that can be cultured into a whole plant.

“Linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome. Recombination between linked loci does not occur during meiosis with high frequency, e.g., linked loci cosegregate at least about 90% of the time, e.g., 91%, 92%, 93%, 94%, 95%, 96%, 97%, 98%, 99%, 99.5%, 99.75%, or more of the time.

“Locus” a chromosome region where a polymorphic nucleic acid, trait determinant, gene, or marker is located. A “gene locus” is a specific chromosome location in the genome of a species where a specific gene can be found.

“Marker Assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

“Marker Assisted Selection” (MAS) is a process by which phenotypes are selected based on marker genotypes.

“Molecular phenotype” is a phenotype detectable at the level of a population of one or more molecules. Such molecules can be nucleic acids, proteins, or metabolites. A molecular phenotype could be an expression profile for one or more gene products, e.g., at a specific stage of plant development, in response to an environmental condition or stress, etc.

“Nucleic acid” refers to any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety). The present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“Percent identity” or “% identity” means the extent to which two optimally aligned polynucleotide segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence.

“Phenotype” refers to the detectable characteristics of a cell or organism which can be influenced by genotype.

“Polymorphism” refers to the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general breeding programs and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a resistance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms. Variations in the DNA sequences of e.g. humans or plants can affect how they handle diseases, bacteria, viruses, chemicals, drugs, etc.

A “population” refers to a set comprising any number, including one, of individuals, objects, or data from which samples are taken for evaluation. Most commonly, the terms relate to a breeding population from which members are selected and crossed to produce progeny in a breeding program. A population can include the progeny of a single breeding cross or a plurality of breeding crosses. The population members need not be identical to the population members selected for use in subsequent cycles of analyses or those ultimately selected to obtain final progeny. Often, a population is derived from a single biparental cross, but may also derive from two or more crosses between the same or different parents. Although a population may comprise any number of individuals, those of skill in the art will recognize that breeders commonly use population sizes ranging from one or two hundred individuals to several thousand, and that the highest performing 5-20% of a population is what is commonly selected to be used in subsequent crosses in order to improve the performance of subsequent generations of the population.

“Primer” refers to an oligonucleotide capable of hybridizing to a target nucleotide sequence to prime the synthesis of DNA by a polymerase. Oligonucleotide primers of the invention can be used in PCR methods and other methods involving nucleic acid amplification. A primer may comprise a “primer tail” which refers to a portion of the primer oligonucleotide sequence which does not hybridize with the target nucleotide sequence.

“Tagging” refers to the addition of a detection label to a nucleic acid sample in order to distinguish it from a second or further nucleic acid sample. Tagging can be performed e.g. by the addition of a sequence identifier or by any other means known in the art. Such sequence identifier can be e.g. a unique base sequence of varying but defined length uniquely used for identifying a specific nucleic acid sample. Typical examples thereof are, for example, ZIP sequences. Using such tag, the origin of a sample can be determined upon further processing. In case of combining processed products originating from different nucleic acid samples, the different nucleic acid samples can be identified using different tags. A “tagged library” refers to a library of tagged nucleic acids.

“Target DNA region” refers to a segment of genomic DNA of one or more nucleotides in length that may or may not be polymorphic in a population.

“Target polymorphism” refers to a specific genomic locus that is known to exhibit one or more variations of a nucleic acid sequence in a population.

“Test sample nucleic acid” refers to a nucleic acid sample that is investigated for polymorphisms.

### EXAMPLES

### Example 1. Multiplex PCR Amplification of 150 Genomic Loci

A set of 150 genomic regions of the cattle genome (Table 1) were amplified by PCR in a multiplex reaction comprising the following reagents.

| | | |
|---|---|---|
| 5X Phusion HF buffer with 7.5 mM | 6.0 | μl |
| MgCl₂ (Thermo Scientific) | | |
| 10 mM dNTP (Agilent) | 0.6 | μl |
| 1X primer (combination of 150 primer pairs) | 3.0 | μl |
| 100% DMSO (Thermo Scientific) | 0.3 | μl |
| Purified BSA 100x (BioLabs) | 0.3 | μl |
| Phusion Taq (2 U/μl) (Thermo Scientific) | 1.0 | μl |
| DNA | 100 | ng |

| | |
|---|---|
| H₂O | to total volume of 30 μl |

The multiplex PCR mixture was amplified under the following conditions.

1. 98° C.—10 min

2. 98° C.—2 min

3. 72° C.—1 min

4. Go to step 2, 9 more times (10 cycles total)

5. 72° C.—10 min

6. Hold at 25° C.

Each primer pair used in the reaction comprises a sequence that binds specifically to a region upstream or downstream of a polymorphism of interest as shown in the Forward Primer and Reverse Primer columns of Table 1. Each forward primer sequence further comprises a tail having a sequence of 5′ ACACGACGCTCTTCCGATCT 3′ (SEQ ID NO: 301) at the 5′ end. Each reverse primer sequence further comprises a tail having a sequence of 5′ CTGAACCCTTGTCGCCATTC 3′ (SEQ ID NO: 302) on the 5′ end.

Concentrations of each primer pair were adjusted individually based on the following regression equation:

$${log}_{10} ( \frac{{reads}_{i} * 100}{{\sum }_{i} {reads}_{i}} ) = 0.34678 + 1.42626 {log}_{10} ( {dilution}_{i} )$$

The equation was developed by counting the number of sequencing reads obtained for all primer pairs, at different concentrations. In the equation, reads stands for the number of reads sequenced for primer i and dilution stands for the concentration level used in the experiment for the same primer i. The equation was used to calculate a primer dilution for a given locus, which is equal to the 1/number of loci to be amplified in the multiplex PCR reaction. Therefore, if amplifying 100 loci, then the primer pairs used to amplify one locus are diluted 1/100. After the outcome of a sequencing run is obtained, the number of reads obtained for primer i, as well as the sum of reads obtained for all primer pairs was plugged into the left side of the formula. Based on that number a new dilution was generated (right side of the formula). That new dilution was then used in future experiments as the final dilution to be used for that pair of primers.

After the first amplification step, amplified DNA was separated from the reaction mixture using AMPure beads, using the following procedure:
30 μl of room temperature AMPure beads (Beckman Coulter, Inc.) were added to each reaction mixture and mixed well
Reaction mixtures and beads were incubate at room temperature for 15 minutes
Reaction mixtures and beads were placed on a magnetic stand for 5 minutes
Liquid was carefully pipetted off
200 μl of freshly prepared 80% ethanol was added and allowed to stand for 30 seconds
Liquid was carefully removed and wash was repeated
After all liquid was removed, beads were allowed to incubate at room temperature for 10 minutes
22 μl of elution buffer were added and mixed well by pipetting
The mixture was allowed to stand for 5 minutes at room temperature
The mixture was placed on a magnetic stand for 5 minutes
20 μl of liquid was carefully removed to a new tube

After the separation step, the product of the first amplification step was further amplified in a second PCR step using a pair of universal primers that bind to the tail of each primer pair used in the first PCR step. Universal Primer 1 had SEQ ID NO: 303 (5′ AATGATACGGCGACCACCGAGATCTACACNNNNNNNNACACTCTTTCCCTACACGA CGCTCTTCCGATCT 3′) and Universal Primer 2 had SEQ ID NO: 304 (5′ CAAGCAGAAGACGGCATACGAGATNNNNNNNNCGGTCTCGGCATTCCTGCTGAACC CTTGTCGCCATTC 3′), where NNNNNNNN represents an optional index (e.g., bar code) that can be inserted into such primers or other primers prepared according to the invention representing the nucleotides of the index used to identify the sample being processed. The second PCR step included the following reagents and was carried out under the conditions described below. Reagent concentration and sources are the same as those described above for the first PCR step.

| | |
|---|---|
| 5X Phusion HF buffer | 10.0 μl |
| 10 mM dNTP | 0.6 μl |
| 10 uM Primer 1 | 1.0 μl |
| 10 uM Primer2 | 1.0 μl |
| Product of first PCR reaction | 20 μl |
| DMSO | 0.5 μl |
| BSA | 0.5 μl |
| Phusion Taq | 1.0 μl |
| H₂O | 15.4 μl |
| Total | 50 μl |

The PCR mixture for the second amplification step was amplified under the following conditions:

1. 98° C.—10 min

2. 98° C.—2 min

3. 57° C.—30 sec

4. 72° C.—30 sec

5. Go to step 2, 9 more times (10 cycles total)

6. 72° C.—10 min

7. Hold at 25° C.

After the second amplification step, amplified DNA was separated from the reaction mixture using a Macherey-Nagel NucleoSpin Gel and PCR Clean-up Kit (Clontech) according to the following procedure:
Reactions from one or multiple samples were combined into one tube and volume was measured (or calculated)
200 μl NTI buffer were added for every 100 μl PCR reaction and mixed (e.g. 1.75 ml PCR and 3.5 ml NTI)
The mixture was added to a column 700 μl at a time and put under vacuum or spun at 11,000 g
After last addition of reaction, the column was washed twice with 700 μl of NT3
The membrane was dried by placing the column at 70° C. for 5 minutes
30 μl of warm NE was added to the column and incubated for 5 minutes
The column was spun

The resulting product corresponded to the DNA library of an individual, containing amplification products of each of 150 regions of the cattle genome. DNA libraries of 24 individuals were quantified, and pooled in equimolar amounts, for sequencing in a HiSeq2500 Illumina DNA sequencer.

### Example 2. Consistency of Reads Across 150 Genomic Loci

The outcome of the sequencing and analysis of a DNA library from one individual according to Example 1 is presented in Table 2. The table shows the average percentage of reads obtained for each of 150 loci relative to the total number of reads produced in the sample, and the standard deviation of 24 replicates. These data demonstrated that the target region was successfully amplified at all target loci, and that the number of reads produced was well-balanced across loci and across replicates.

TABLE 1
Cattle genome loci (identified by the International Society for Animal Genetics label) and
genomic regions amplified by the amplification method of the present invention (identified by
the Bos taurus genome assembly 3.1), and primer pairs used for the amplification.

| | | Forward | Reverse |
|---|---|---|---|
| | | Primer | Primer |
| ISAG_LABEL | REGION_UMD_3.1 | (SEQ ID NO) | (SEQ ID NO) |
| ARS-USMARC-Parent-EF026087- | gi\|258513354\|ref\|AC_000170.1\|: 1982204 . . . 1982214 | 1 | 2 |
| rs29011643 | | | |
| ARS-USMARC-Parent-DQ846693- | gi\|258513351\|ref\|AC_000173.1\|: 9855271 . . . 9855281 | 3 | 4 |
| rs29017621 | | | |
| ARS-BFGL-NGS-38423 | gi\|258513364\|ref\|AC_000160.1\|: 12709849 . . . 12709859 | 5 | 6 |
| ARS-BFGL-NGS-38620 | gi\|258513349\|ref\|AC_000175.1\|: 64382700 . . . 64382710 | 7 | 8 |
| ARS-USMARC-Parent-AY853302- | gi\|258513354\|ref\|AC_000170.1\|: 47397982 . . . 47397992 | 9 | 10 |
| no-rs | | | |
| ARS-USMARC-Parent-AY919868- | gi\|258513347\|ref\|AC_000177.1\|: 46066104 . . . 46066114 | 11 | 12 |
| rs29002211 | | | |
| ARS-USMARC-Parent-EF026085- | gi\|258513346\|ref\|AC_000178.1\|: 65198291 . . . 65198301 | 13 | 14 |
| rs29021607 | | | |
| UA-IFASA-5034 | gi\|258513338\|ref\|AC_000186.1\|: 29052618 . . . 29052628 | 15 | 16 |
| ARS-USMARC-Parent-DQ995976- | gi\|258513346\|ref\|AC_000178.1\|: 3088881 . . . 3088891 | 17 | 18 |
| no-rs | | | |
| ARS-BFGL-NGS-112094 | gi\|258513354\|ref\|AC_000170.1\|: 69187737 . . . 69187747 | 19 | 20 |
| Hapmap47281-BTA-40051 | gi\|258513351\|ref\|AC_000173.1\|: 72931870 . . . 72931880 | 21 | 22 |
| ARS-USMARC-Parent-AY842473- | gi\|258513364\|ref\|AC_000160.1\|: 49703642 . . . 49703652 | 23 | 24 |
| rs29001956 | | | |
| ARS-USMARC-Parent-EF034084- | gi\|258513340\|ref\|AC_000184.1\|: 21480565 . . . 21480575 | 25 | 26 |
| rs29016185 | | | |
| ARS-BFGL-NGS-26517 | gi\|258513346\|ref\|AC_000178.1\|: 52139178 . . . 52139188 | 27 | 28 |
| Hapmap40729-BTA-40319 | gi\|258513351\|ref\|AC_000173.1\|: 19882678 . . . 19882688 | 29 | 30 |
| ARS-USMARC-Parent-DQ786759- | gi\|258513360\|ref\|AC_000164.1\|: 94259467 . . . 94259477 | 31 | 32 |
| rs29026696 | | | |
| ARS-USMARC-Parent-DQ404149- | gi\|258517435\|ref\|AC_000158.1\|: 99314920 . . . 99314930 | 33 | 34 |
| no-rs | | | |
| ARS-USMARC-Parent-DQ647190- | gi\|258513361\|ref\|AC_000163.1\|: 13897063 . . . 13897073 | 35 | 36 |
| rs29013632 | | | |
| ARS-USMARC-Parent-EF093511- | gi\|258513346\|ref\|AC_000178.1\|: 26620008 . . . 26620018 | 37 | 38 |
| rs29012316 | | | |
| BTA-73768-no-rs | gi\|258513362\|ref\|AC_000162.1\|: 67031018 . . . 67031028 | 39 | 40 |
| BTB-01902778 | gi\|258513361\|ref\|AC_000163.1\|: 114165528 . . . 114165538 | 41 | 42 |
| ARS-USMARC-Parent-AY850194- | gi\|258513359\|ref\|AC_000165.1\|: 59996426 . . . 59996436 | 43 | 44 |
| no-rs | | | |
| Hapmap36588- | gi\|258513347\|ref\|AC_000177.1\|: 2289044 . . . 2289054 | 45 | 46 |
| SCAFFOLD90561_9460 | | | |
| ARS-USMARC-Parent-EF034082- | gi\|258513345\|ref\|AC_000179.1\|: 56526457 . . . 56526467 | 47 | 48 |
| rs29013532 | | | |
| Hapmap40148-BTA-92999 | gi\|258513356\|ref\|AC_000168.1\|: 36713466 . . . 36713476 | 49 | 50 |
| ARS-BFGL-NGS-106015 | gi\|258513361\|ref\|AC_000163.1\|: 61199567 . . . 61199577 | 51 | 52 |
| ARS-BFGL-NGS-99210 | gi\|258513350\|ref\|AC_000174.1\|: 52740913 . . . 52740923 | 53 | 54 |
| BTB-01980499 | gi\|258513355\|ref\|AC_000169.1\|: 61345449 . . . 61345459 | 55 | 56 |
| Hapmap54020-rs29023153 | gi\|258513349\|ref\|AC_000175.1\|: 6776527 . . . 6776537 | 57 | 58 |
| ARS-BFGL-NGS-14740 | gi\|258513358\|ref\|AC_000166.1\|: 14436980 . . . 14436990 | 59 | 60 |
| ARS-USMARC-Parent-DQ888313- | gi\|258513347\|ref\|AC_000177.1\|: 17837670 . . . 17837680 | 61 | 62 |
| no-rs | | | |
| ARS-USMARC-Parent-DQ990832- | gi\|258513345\|ref\|AC_000179.1\|: 11038200 . . . 11038210 | 63 | 64 |
| rs29015065 | | | |
| ARS-USMARC-Parent-AY844963- | gi\|258513362\|ref\|AC_000162.1\|: 98102344 . . . 98102354 | 65 | 66 |
| rs17871338 | | | |
| ARS-USMARC-Parent-DQ888311- | gi\|258513348\|ref\|AC_000176.1\|: 8505312 . . . 8505322 | 67 | 68 |
| rs29017313 | | | |
| ARS-USMARC-Parent-DQ786764- | gi\|258513355\|ref\|AC_000169.1\|: 25668969 . . . 25668979 | 69 | 70 |
| no-rs | | | |
| ARS-USMARC-Parent-DQ888309- | gi\|258513360\|ref\|AC_000164.1\|: 8272789 . . . 8272799 | 71 | 72 |
| rs29013741 | | | |
| ARS-USMARC-Parent-AY842472- | gi\|258513364\|ref\|AC_000160.1\|: 40399131 . . . 40399141 | 73 | 74 |
| rs29001941 | | | |
| BTB-01371672 | gi\|258513352\|ref\|AC_000172.1\|: 50566480 . . . 50566490 | 75 | 76 |
| ARS-USMARC-Parent-DQ846691- | gi\|258513353\|ref\|AC_000171.1\|: 48380424 . . . 48380434 | 77 | 78 |
| rs29019814 | | | |
| ARS-USMARC-Parent-EF034081- | gi\|258513354\|ref\|AC_000170.1\|: 25606464 . . . 25606474 | 79 | 80 |
| rs29009668 | | | |
| ARS-BFGL-NGS-31807 | gi\|258513357\|ref\|AC_000167.1\|: 61684699 . . . 61684709 | 81 | 82 |
| ARS-BFGL-NGS-111114 | gi\|258513357\|ref\|AC_000167.1\|: 84446637 . . . 84446647 | 83 | 84 |
| ARS-BFGL-NGS-111076 | gi\|258513346\|ref\|AC_000178.1\|: 21220443 . . . 21220453 | 85 | 86 |
| Hapmap59876-rs29018046 | gi\|258513365\|ref\|AC_000159.1\|: 13853724 . . . 13853734 | 87 | 88 |
| ARS-USMARC-Parent-DQ786763- | gi\|258513355\|ref\|AC_000169.1\|: 11824648 . . . 11824658 | 89 | 90 |
| rs29020472 | | | |
| ARS-BFGL-NGS-15731 | gi\|258513360\|ref\|AC_000164.1\|: 96936140 . . . 96936150 | 91 | 92 |
| ARS-USMARC-Parent-DQ381153- | gi\|258517435\|ref\|AC_000158.1\|: 3249052 . . . 3249062 | 93 | 94 |
| rs29012842 | | | |
| ARS-BFGL-NGS-118319 | gi\|258513350\|ref\|AC_000174.1\|: 9279318 . . . 9279328 | 95 | 96 |
| ARS-USMARC-Parent-AY943841- | gi\|258517435\|ref\|AC_000158.1\|: 138583178 . . . 138583188 | 97 | 98 |
| rs17871566 | | | |
| ARS-BFGL-NGS-101456 | gi\|258513360\|ref\|AC_000164.1\|: 82825192 . . . 82825202 | 99 | 100 |
| ARS-BFGL-NGS-118340 | gi\|258513349\|ref\|AC_000175.1\|: 26844469 . . . 26844479 | 101 | 102 |
| Hapmap55441-rs29010990 | gi\|258513359\|ref\|AC_000165.1\|: 103137117 . . . 103137127 | 103 | 104 |
| ARS-USMARC-Parent-DQ647189- | gi\|258513362\|ref\|AC_000162.1\|: 63273381 . . . 63273391 | 105 | 106 |
| rs29012226 | | | |
| Hapmap51227-BTA-41809 | gi\|258513350\|ref\|AC_000174.1\|: 67216415 . . . 67216425 | 107 | 108 |
| ARS-BFGL-NGS-111053 | gi\|258513362\|ref\|AC_000162.1\|: 72264598 . . . 72264608 | 109 | 110 |
| ARS-USMARC-Parent-AY841151- | gi\|258513365\|ref\|AC_000159.1\|: 45832882 . . . 45832892 | 111 | 112 |
| rs29003466 | | | |
| ARS-BFGL-NGS-39978 | gi\|258513365\|ref\|AC_000159.1\|: 5757350 . . . 5757360 | 113 | 114 |
| ARS-USMARC-Parent-DQ786762- | gi\|258513357\|ref\|AC_000167.1\|: 81572247 . . . 81572257 | 115 | 116 |
| rs29010772 | | | |
| ARS-USMARC-Parent-DQ786765- | gi\|258513358\|ref\|AC_000166.1\|: 98483341 . . . 98483351 | 117 | 118 |
| rs29009858 | | | |
| ARS-BFGL-NGS-11383 | gi\|258513350\|ref\|AC_000174.1\|: 12891992 . . . 12892002 | 119 | 120 |
| BTA-37062-no-rs | gi\|258513352\|ref\|AC_000172.1\|: 51528612 . . . 51528622 | 121 | 122 |
| ARS-USMARC-Parent-EF034085- | gi\|258513339\|ref\|AC_000185.1\|: 5913221 . . . 5913231 | 123 | 124 |
| rs29025677 | | | |
| ARS-BFGL-NGS-10035 | gi\|258513341\|ref\|AC_000183.1\|: 39952149 . . . 39952159 | 125 | 126 |
| ARS-USMARC-Parent-AY860426- | gi\|258513350\|ref\|AC_000174.1\|: 56512514 . . . 56512524 | 127 | 128 |
| no-rs | | | |
| UA-IFASA-6532 | gi\|258513345\|ref\|AC_000179.1\|: 21770144 . . . 21770154 | 129 | 130 |
| ARS-USMARC-Parent-AY851162- | gi\|258513356\|ref\|AC_000168.1\|: 46411095 . . . 46411105 | 131 | 132 |
| no-rs | | | |
| ARS-USMARC-Parent-DQ916057- | gi\|258513360\|ref\|AC_000164.1\|: 81591567 . . . 81591607 | 133 | 134 |
| rs29009979 | | | |
| BTA-100621-no-rs | gi\|258513345\|ref\|AC_000179.1\|: 25598775 . . . 25598785 | 135 | 136 |
| ARS-BFGL-NGS-93119 | gi\|258513358\|ref\|AC_000166.1\|: 28149126 . . . 28149136 | 137 | 138 |
| ARS-USMARC-Parent-DQ984826- | gi\|258513353\|ref\|AC_000171.1\|: 27751883 . . . 27751893 | 139 | 140 |
| rs29027559 | | | |
| ARS-USMARC-Parent-AY853303- | gi\|258513354\|ref\|AC_000170.1\|: 75383369 . . . 75383379 | 141 | 142 |
| no-rs | | | |
| ARS-USMARC-Parent-DQ674265- | gi\|258513359\|ref\|AC_000165.1\|: 106174866 . . . 106174876 | 143 | 144 |
| rs29011266 | | | |
| ARS-USMARC-Parent-EF034087- | gi\|258513339\|ref\|AC_000185.1\|: 16097744 . . . 16097754 | 145 | 146 |
| no-rs | | | |
| ARS-USMARC-Parent-AY863214- | gi\|258513349\|ref\|AC_000175.1\|: 46647172 . . . 46647182 | 147 | 148 |
| rs17871744 | | | |
| ARS-USMARC-Parent-DQ837643- | gi\|258513356\|ref\|AC_000168.1\|: 66341584 . . . 66341594 | 149 | 150 |
| rs29018818 | | | |
| Hapmap39461-BTA-109898 | gi\|258513346\|ref\|AC_000178.1\|: 27927776 . . . 27927786 | 151 | 152 |
| ARS-USMARC-Parent-AY842474- | gi\|258513364\|ref\|AC_000160.1\|: 51976641 . . . 51976651 | 153 | 154 |
| rs29003226 | | | |
| ARS-USMARC-Parent-AY941204- | gi\|258513342\|ref\|AC_000182.1\|: 14683146 . . . 14683156 | 155 | 156 |
| rs17872131 | | | |
| ARS-USMARC-Parent-EF042091- | gi\|258513339\|ref\|AC_000185.1\|: 44261940 . . . 44261950 | 157 | 158 |
| rs29014974 | | | |
| ARS-USMARC-Parent-AY858890- | gi\|258513350\|ref\|AC_000174.1\|: 29936152 . . . 29936162 | 159 | 160 |
| rs29002256 | | | |
| ARS-USMARC-Parent-DQ500958- | gi\|258513362\|ref\|AC_000162.1\|: 27825113 . . . 27825123 | 161 | 162 |
| no-rs | | | |
| ARS-USMARC-569 | gi\|258513350\|ref\|AC_000174.1\|: 30360937 . . . 30360947 | 163 | 164 |
| ARS-BFGL-NGS-42505 | gi\|258513353\|ref\|AC_000171.1\|: 62478237 . . . 62478247 | 165 | 166 |
| ARS-USMARC-Parent-AY939849- | gi\|258513343\|ref\|AC_000181.1\|: 56415789 . . . 56415799 | 167 | 168 |
| rs17870274 | | | |
| ARS-BFGL-NGS-70946 | gi\|258513357\|ref\|AC_000167.1\|: 14574448 . . . 14574458 | 169 | 170 |
| ARS-USMARC-Parent-EF164803- | gi\|258513348\|ref\|AC_000176.1\|: 55174255 . . . 55174265 | 171 | 172 |
| rs29011141 | | | |
| ARS-USMARC-Parent-DQ786758- | gi\|258513360\|ref\|AC_000164.1\|: 18454631 . . . 18454641 | 173 | 174 |
| rs29024430 | | | |
| ARS-USMARC-Parent-EF028073- | gi\|258513349\|ref\|AC_000175.1\|: 1839728 . . . 1839738 | 175 | 176 |
| rs29014953 | | | |
| ARS-USMARC-Parent-AY916666- | gi\|258513348\|ref\|AC_000176.1\|: 44799385 . . . 44799395 | 177 | 178 |
| no-rs | | | |
| ARS-USMARC-Parent-AY914316- | gi\|258513349\|ref\|AC_000175.1\|: 48812009 . . . 48812019 | 179 | 180 |
| rs17871403 | | | |
| ARS-USMARC-Parent-EF034086- | gi\|258513341\|ref\|AC_000183.1\|: 38233332 . . . 38233342 | 181 | 182 |
| no-rs | | | |
| ARS-USMARC-Parent-DQ786766- | gi\|258513357\|ref\|AC_000167.1\|: 3530266 . . . 3530276 | 183 | 184 |
| rs29012070 | | | |
| ARS-USMARC-Parent-DQ650636- | gi\|258513359\|ref\|AC_000165.1\|: 28799244 . . . 28799254 | 185 | 186 |
| rs29024525 | | | |
| ARS-BFGL-NGS-67146 | gi\|258513365\|ref\|AC_000159.1\|: 118773628 . . . 118773638 | 187 | 188 |
| Hapmap24215-BTA-163266 | gi\|258513340\|ref\|AC_000184.1\|: 10764820 . . . 10764830 | 189 | 190 |
| ARS-BFGL-NGS-96125 | gi\|258513347\|ref\|AC_000177.1\|: 58449207 . . . 58449217 | 191 | 192 |
| ARS-BFGL-NGS-15506 | gi\|258513362\|ref\|AC_000162.1\|: 88334671 . . . 88334681 | 193 | 194 |
| ARS-BFGL-NGS-36513 | gi\|258513348\|ref\|AC_000176.1\|: 17236702 . . . 17236712 | 195 | 196 |
| ARS-USMARC-Parent-DQ647187- | gi\|258513364\|ref\|AC_000160.1\|: 21146872 . . . 21146882 | 197 | 198 |
| rs29010510 | | | |
| Hapmap31098-BTA-136127 | gi\|258513346\|ref\|AC_000178.1\|: 62028403 . . . 62028413 | 199 | 200 |
| ARS-USMARC-Parent-DQ866817- | gi\|258513352\|ref\|AC_000172.1\|: 38078770 . . . 38078780 | 201 | 202 |
| no-rs | | | |
| ARS-USMARC-Parent-DQ647186- | gi\|258513363\|ref\|AC_000161.1\|: 17200589 . . . 17200599 | 203 | 204 |
| rs29014143 | | | |
| Hapmap35535- | gi\|258513344\|ref\|AC_000180.1\|: 41700824 . . . 41700834 | 205 | 206 |
| SCAFFOLD86180_8791 | | | |
| ARS-USMARC-Parent-DQ404152- | gi\|258513365\|ref\|AC_000159.1\|: 5306833 . . . 5306843 | 207 | 208 |
| rs29022245 | | | |
| ARS-USMARC-Parent-DQ916058- | gi\|258513359\|ref\|AC_000165.1\|: 1554701 . . . 1554711 | 209 | 210 |
| rs29016146 | | | |
| ARS-BFGL-NGS-119662 | gi\|258513361\|ref\|AC_000163.1\|: 118292323 . . . 118292333 | 211 | 212 |
| ARS-USMARC-Parent-DQ984825- | gi\|258513357\|ref\|AC_000167.1\|: 98230474 . . . 98230484 | 213 | 214 |
| rs29012457 | | | |
| ARS-USMARC-Parent-EF026084- | gi\|258513348\|ref\|AC_000176.1\|: 15345307 . . . 15345317 | 215 | 216 |
| rs29025380 | | | |
| ARS-USMARC-Parent-DQ837645- | gi\|258513356\|ref\|AC_000168.1\|: 24553002 . . . 24553012 | 217 | 218 |
| rs29015870 | | | |
| ARS-USMARC-Parent-EF026086- | gi\|258513339\|ref\|AC_000185.1\|: 35331555 . . . 35331565 | 219 | 220 |
| rs29013660 | | | |
| BTB-00818821 | gi\|258513346\|ref\|AC_000178.1\|: 40408798 . . . 40408808 | 221 | 222 |
| ARS-BFGL-NGS-57711 | gi\|258513348\|ref\|AC_000176.1\|: 40692424 . . . 40692434 | 223 | 224 |
| ARS-USMARC-Parent-DQ846688- | gi\|258513362\|ref\|AC_000162.1\|: 119261604 . . . 119261614 | 225 | 226 |
| rs29023691 | | | |
| ARS-USMARC-Parent-DQ789028- | gi\|258513361\|ref\|AC_000163.1\|: 46936177 . . . 46936187 | 227 | 228 |
| rs29017713 | | | |
| ARS-USMARC-Parent-DQ470475- | gi\|258513362\|ref\|AC_000162.1\|: 7651048 . . . 7651058 | 229 | 230 |
| no-rs | | | |
| ARS-USMARC-Parent-AY937242- | gi\|258513344\|ref\|AC_000180.1\|: 27306790 . . . 27306800 | 231 | 232 |
| rs17872223 | | | |
| ARS-USMARC-Parent-EF034083- | gi\|258513342\|ref\|AC_000182.1\|: 3126433 . . . 3126443 | 233 | 234 |
| rs29018286 | | | |
| ARS-USMARC-Parent-AY842475- | gi\|258513363\|ref\|AC_000161.1\|: 20181744 . . . 20181754 | 235 | 236 |
| rs29002127 | | | |
| ARS-BFGL-NGS-55943 | gi\|258513352\|ref\|AC_000172.1\|: 64628701 . . . 64628711 | 237 | 238 |
| ARS-USMARC-Parent-DQ404150- | gi\|258517435\|ref\|AC_000158.1\|: 59409833 . . . 59409843 | 239 | 240 |
| rs29012530 | | | |
| ARS-USMARC-Parent-DQ404153- | gi\|258513338\|ref\|AC_000186.1\|: 44756497 . . . 44756507 | 241 | 242 |
| no-rs | | | |
| ARS-USMARC-Parent-EF150946- | gi\|258513341\|ref\|AC_000183.1\|: 13229214 . . . 13229224 | 243 | 244 |
| rs29023666 | | | |
| ARS-BFGL-NGS-102169 | gi\|258513340\|ref\|AC_000184.1\|: 12930431 . . . 12930441 | 245 | 246 |
| Hapmap42648-BTA-71195 | gi\|258513363\|ref\|AC_000161.1\|: 71061219 . . . 71061229 | 247 | 248 |
| ARS-USMARC-Parent-DQ435443- | gi\|258513364\|ref\|AC_000160.1\|: 58040465 . . . 58040475 | 249 | 250 |
| rs29010802 | | | |
| ARS-USMARC-Parent-DQ404151- | gi\|258517435\|ref\|AC_000158.1\|: 151349509 . . . 151349519 | 251 | 252 |
| rs29019282 | | | |
| ARS-USMARC-Parent-EF089234- | gi\|258513344\|ref\|AC_000180.1\|: 50884047 . . . 50884057 | 253 | 254 |
| rs29020870 | | | |
| ARS-USMARC-Parent-DQ846692- | gi\|258513353\|ref\|AC_000171.1\|: 80082918 . . . 80082928 | 255 | 256 |
| rs29010281 | | | |
| ARS-BFGL-NGS-112325 | gi\|258513355\|ref\|AC_000169.1\|: 79643098 . . . 79643108 | 257 | 258 |
| Hapmap41591-BTA-59790 | gi\|258513342\|ref\|AC_000182.1\|: 27912097 . . . 27912107 | 259 | 260 |
| ARS-BFGL-NGS-115514 | gi\|258513352\|ref\|AC_000172.1\|: 38003129 . . . 38003139 | 261 | 262 |
| ARS-USMARC-Parent-DQ990834- | gi\|258513341\|ref\|AC_000183.1\|: 8221265 . . . 8221275 | 263 | 264 |
| rs29013727 | | | |
| ARS-USMARC-Parent-EF042090- | gi\|258513352\|ref\|AC_000172.1\|: 21207524 . . . 21207534 | 265 | 266 |
| no-rs | | | |
| Hapmap43057-BTA-80741 | gi\|258513360\|ref\|AC_000164.1\|: 13279967 . . . 13279977 | 267 | 268 |
| ARS-USMARC-Parent-DQ916059- | gi\|258513349\|ref\|AC_000175.1\|: 23426209 . . . 23426219 | 269 | 270 |
| rs29009907 | | | |
| ARS-BFGL-NGS-117322 | gi\|258513356\|ref\|AC_000168.1\|: 77884739 . . . 77884749 | 271 | 272 |
| ARS-USMARC-Parent-AY856094- | gi\|258513338\|ref\|AC_000186.1\|: 9160934 . . . 9160944 | 273 | 274 |
| rs17871190 | | | |
| ARS-BFGL-NGS-42283 | gi\|258513356\|ref\|AC_000168.1\|: 95543406 . . . 95543416 | 275 | 276 |
| ARS-USMARC-Parent-DQ846690- | gi\|258513353\|ref\|AC_000171.1\|: 10171914 . . . 10171924 | 277 | 278 |
| no-rs | | | |
| ARS-BFGL-NGS-24419 | gi\|258513345\|ref\|AC_000179.1\|: 11756778 . . . 11756788 | 279 | 280 |
| ARS-BFGL-NGS-31640 | gi\|258513358\|ref\|AC_000166.1\|: 52430559 . . . 52430569 | 281 | 282 |
| ARS-USMARC-Parent-AY929334- | gi\|258513344\|ref\|AC_000180.1\|: 7219970 . . . 7219980 | 283 | 284 |
| no-rs | | | |
| ARS-USMARC-Parent-EF141102- | gi\|258513340\|ref\|AC_000184.1\|: 37513918 . . . 37513928 | 285 | 286 |
| rs29015783 | | | |
| ARS-BFGL-BAC-19454 | gi\|258513351\|ref\|AC_000173.1\|: 13695206 . . . 13695216 | 287 | 288 |
| ARS-USMARC-Parent-DQ489377- | gi\|258513364\|ref\|AC_000160.1\|: 98188379 . . . 98188389 | 289 | 290 |
| rs29026932 | | | |
| ARS-USMARC-Parent-DQ990833- | gi\|258513343\|ref\|AC_000181.1\|: 15447766 . . . 15447776 | 291 | 292 |
| rs29010147 | | | |
| Hapmap54547-rs29012198 | gi\|258513363\|ref\|AC_000161.1\|: 110776619 . . . 110776629 | 293 | 294 |
| Hapmap50598-BTA-122724 | gi\|258513348\|ref\|AC_000176.1\|: 4670551 . . . 4670561 | 295 | 296 |
| ARS-USMARC-Parent-AY851163- | gi\|258513356\|ref\|AC_000168.1\|: 103047469 . . . 103047479 | 297 | 298 |
| rs17871661 | | | |
| ARS-BFGL-NGS-86662 | gi\|258517435\|ref\|AC_000158.1\|: 40206012 . . . 40206022 | 299 | 300 |

TABLE 2
Percentage of reads that originated from each of 150 loci in the cattle genome relative to
total reads in the sample obtained using the amplification method provided herein. Average
percentage as well as standard deviation between 24 replicates for each locus are shown.

| ISAG_LABEL | REGION_UMD_3.1 | Avg. | SD |
|---|---|---|---|
| ARS-USMARC-Parent-EF026087-rs29011643 | gi\|258513354\|ref\|AC_000170.1\|: 1982204 . . . 1982214 | 0.102 | 0.082 |
| ARS-USMARC-Parent-DQ846693- | gi\|258513351\|ref\|AC_000173.1\|: 9855271 . . . 9855281 | 0.116 | 0.038 |
| rs29017621 | | | |
| ARS-BFGL-NGS-38423 | gi\|258513364\|ref\|AC_000160.1\|: 12709849 . . . 12709859 | 0.119 | 0.034 |
| ARS-BFGL-NGS-38620 | gi\|258513349\|ref\|AC_000175.1\|: 64382700 . . . 64382710 | 0.122 | 0.035 |
| ARS-USMARC-Parent-AY853302-no-rs | gi\|258513354\|ref\|AC_000170.1\|: 47397982 . . . 47397992 | 0.128 | 0.062 |
| ARS-USMARC-Parent-AY919868-rs29002211 | gi\|258513347\|ref\|AC_000177.1\|: 46066104 . . . 46066114 | 0.129 | 0.075 |
| ARS-USMARC-Parent-EF026085-rs29021607 | gi\|258513346\|ref\|AC_000178.1\|: 65198291 . . . 65198301 | 0.132 | 0.145 |
| UA-IFASA-5034 | gi\|258513338\|ref\|AC_000186.1\|: 29052618 . . . 29052628 | 0.133 | 0.049 |
| ARS-USMARC-Parent-DQ995976-no-rs | gi\|258513346\|ref\|AC_000178.1\|: 3088881 . . . 3088891 | 0.136 | 0.121 |
| ARS-BFGL-NGS-112094 | gi\|258513354\|ref\|AC_000170.1\|: 69187737 . . . 69187747 | 0.137 | 0.032 |
| Hapmap47281-BTA-40051 | gi\|258513351\|ref\|AC_000173.1\|: 72931870 . . . 72931880 | 0.143 | 0.027 |
| ARS-USMARC-Parent-AY842473-rs29001956 | gi\|258513364\|ref\|AC_000160.1\|: 49703642 . . . 49703652 | 0.144 | 0.019 |
| ARS-USMARC-Parent-EF034084-rs29016185 | gi\|258513340\|ref\|AC_000184.1\|: 21480565 . . . 21480575 | 0.146 | 0.070 |
| ARS-BFGL-NGS-26517 | gi\|258513346\|ref\|AC_000178.1\|: 52139178 . . . 52139188 | 0.146 | 0.138 |
| Hapmap40729-BTA-40319 | gi\|258513351\|ref\|AC_000173.1\|: 19882678 . . . 19882688 | 0.146 | 0.024 |
| ARS-USMARC-Parent-DQ786759- | gi\|258513360\|ref\|AC_000164.1\|: 94259467 . . . 94259477 | 0.147 | 0.033 |
| rs29026696 | | | |
| ARS-USMARC-Parent-DQ404149-no-rs | gi\|258517435\|ref\|AC_000158.1\|: 99314920 . . . 99314930 | 0.159 | 0.074 |
| ARS-USMARC-Parent-DQ647190- | gi\|258513361\|ref\|AC_000163.1\|: 13897063 . . . 13897073 | 0.162 | 0.067 |
| rs29013632 | | | |
| ARS-USMARC-Parent-EF093511-rs29012316 | gi\|258513346\|ref\|AC_000178.1\|: 26620008 . . . 26620018 | 0.165 | 0.028 |
| BTA-73768-no-rs | gi\|258513362\|ref\|AC_000162.1\|: 67031018 . . . 67031028 | 0.168 | 0.072 |
| BTB-01902778 | gi\|258513361\|ref\|AC_000163.1\|: 114165528 . . . 114165538 | 0.170 | 0.058 |
| ARS-USMARC-Parent-AY850194-no-rs | gi\|258513359\|ref\|AC_000165.1\|: 59996426 . . . 59996436 | 0.171 | 0.048 |
| Hapmap36588-SCAFFOLD90561_9460 | gi\|258513347\|ref\|AC_000177.1\|: 2289044 . . . 2289054 | 0.176 | 0.119 |
| ARS-USMARC-Parent-EF034082-rs29013532 | gi\|258513345\|ref\|AC_000179.1\|: 56526457 . . . 56526467 | 0.179 | 0.091 |
| Hapmap40148-BTA-92999 | gi\|258513356\|ref\|AC_000168.1\|: 36713466 . . . 36713476 | 0.185 | 0.086 |
| ARS-BFGL-NGS-106015 | gi\|258513361\|ref\|AC_000163.1\|: 61199567 . . . 61199577 | 0.199 | 0.054 |
| ARS-BFGL-NGS-99210 | gi\|258513350\|ref\|AC_000174.1\|: 52740913 . . . 52740923 | 0.202 | 0.088 |
| BTB-01980499 | gi\|258513355\|ref\|AC_000169.1\|: 61345449 . . . 61345459 | 0.206 | 0.064 |
| Hapmap54020-rs29023153 | gi\|258513349\|ref\|AC_000175.1\|: 6776527 . . . 6776537 | 0.208 | 0.043 |
| ARS-BFGL-NGS-14740 | gi\|258513358\|ref\|AC_000166.1\|: 14436980 . . . 14436990 | 0.208 | 0.070 |
| ARS-USMARC-Parent-DQ888313-no-rs | gi\|258513347\|ref\|AC_000177.1\|: 17837670 . . . 17837680 | 0.211 | 0.063 |
| ARS-USMARC-Parent-DQ990832- | gi\|258513345\|ref\|AC_000179.1\|: 11038200 . . . 11038210 | 0.215 | 0.113 |
| rs29015065 | | | |
| ARS-USMARC-Parent-AY844963-rs17871338 | gi\|258513362\|ref\|AC_000162.1\|: 98102344 . . . 98102354 | 0.216 | 0.050 |
| ARS-USMARC-Parent-DQ888311- | gi\|258513348\|ref\|AC_000176.1\|: 8505312 . . . 8505322 | 0.219 | 0.063 |
| rs29017313 | | | |
| ARS-USMARC-Parent-DQ786764-no-rs | gi\|258513355\|ref\|AC_000169.1\|: 25668969 . . . 25668979 | 0.221 | 0.106 |
| ARS-USMARC-Parent-DQ888309- | gi\|258513360\|ref\|AC_000164.1\|: 8272789 . . . 8272799 | 0.222 | 0.130 |
| rs29013741 | | | |
| ARS-USMARC-Parent-AY842472-rs29001941 | gi\|258513364\|ref\|AC_000160.1\|: 40399131 . . . 40399141 | 0.223 | 0.146 |
| BTB-01371672 | gi\|258513352\|ref\|AC_000172.1\|: 50566480 . . . 50566490 | 0.225 | 0.151 |
| ARS-USMARC-Parent-DQ846691- | gi\|258513353\|ref\|AC_000171.1\|: 48380424 . . . 48380434 | 0.227 | 0.118 |
| rs29019814 | | | |
| ARS-USMARC-Parent-EF034081-rs29009668 | gi\|258513354\|ref\|AC_000170.1\|: 25606464 . . . 25606474 | 0.231 | 0.065 |
| ARS-BFGL-NGS-31807 | gi\|258513357\|ref\|AC_000167.1\|: 61684699 . . . 61684709 | 0.233 | 0.200 |
| ARS-BFGL-NGS-111114 | gi\|258513357\|ref\|AC_000167.1\|: 84446637 . . . 84446647 | 0.240 | 0.090 |
| ARS-BFGL-NGS-111076 | gi\|258513346\|ref\|AC_000178.1\|: 21220443 . . . 21220453 | 0.241 | 0.044 |
| Hapmap59876-rs29018046 | gi\|258513365\|ref\|AC_000159.1\|: 13853724 . . . 13853734 | 0.245 | 0.149 |
| ARS-USMARC-Parent-DQ786763- | gi\|258513355\|ref\|AC_000169.1\|: 11824648 . . . 11824658 | 0.254 | 0.050 |
| rs29020472 | | | |
| ARS-BFGL-NGS-15731 | gi\|258513360\|ref\|AC_000164.1\|: 96936140 . . . 96936150 | 0.260 | 0.037 |
| ARS-USMARC-Parent-DQ381153- | gi\|258517435\|ref\|AC_000158.1\|: 3249052 . . . 3249062 | 0.262 | 0.064 |
| rs29012842 | | | |
| ARS-BFGL-NGS-118319 | gi\|258513350\|ref\|AC_000174.1\|: 9279318 . . . 9279328 | 0.264 | 0.112 |
| ARS-USMARC-Parent-AY943841-rs17871566 | gi\|258517435\|ref\|AC_000158.1\|: 138583178 . . . 138583188 | 0.264 | 0.080 |
| ARS-BFGL-NGS-101456 | gi\|258513360\|ref\|AC_000164.1\|: 82825192 . . . 82825202 | 0.269 | 0.109 |
| ARS-BFGL-NGS-118340 | gi\|258513349\|ref\|AC_000175.1\|: 26844469 . . . 26844479 | 0.270 | 0.083 |
| Hapmap55441-rs29010990 | gi\|258513359\|ref\|AC_000165.1\|: 103137117 . . . 103137127 | 0.271 | 0.171 |
| ARS-USMARC-Parent-DQ647189- | gi\|258513362\|ref\|AC_000162.1\|: 63273381 . . . 63273391 | 0.273 | 0.080 |
| rs29012226 | | | |
| Hapmap51227-BTA-41809 | gi\|258513350\|ref\|AC_000174.1\|: 67216415 . . . 67216425 | 0.278 | 0.197 |
| ARS-BFGL-NGS-111053 | gi\|258513362\|ref\|AC_000162.1\|: 72264598 . . . 72264608 | 0.278 | 0.036 |
| ARS-USMARC-Parent-AY841151-rs29003466 | gi\|258513365\|ref\|AC_000159.1\|: 45832882 . . . 45832892 | 0.279 | 0.130 |
| ARS-BFGL-NGS-39978 | gi\|258513365\|ref\|AC_000159.1\|: 5757350 . . . 5757360 | 0.283 | 0.055 |
| ARS-USMARC-Parent-DQ786762- | gi\|258513357\|ref\|AC_000167.1\|: 81572247 . . . 81572257 | 0.285 | 0.082 |
| rs29010772 | | | |
| ARS-USMARC-Parent-DQ786765- | gi\|258513358\|ref\|AC_000166.1\|: 98483341 . . . 98483351 | 0.291 | 0.070 |
| rs29009858 | | | |
| ARS-BFGL-NGS-11383 | gi\|258513350\|ref\|AC_000174.1\|: 12891992 . . . 12892002 | 0.296 | 0.085 |
| BTA-37062-no-rs | gi\|258513352\|ref\|AC_000172.1\|: 51528612 . . . 51528622 | 0.297 | 0.173 |
| ARS-USMARC-Parent-EF034085-rs29025677 | gi\|258513339\|ref\|AC_000185.1\|: 5913221 . . . 5913231 | 0.308 | 0.095 |
| ARS-BFGL-NGS-10035 | gi\|258513341\|ref\|AC_000183.1\|: 39952149 . . . 39952159 | 0.309 | 0.111 |
| ARS-USMARC-Parent-AY860426-no-rs | gi\|258513350\|ref\|AC_000174.1\|: 56512514 . . . 56512524 | 0.314 | 0.345 |
| UA-IFASA-6532 | gi\|258513345\|ref\|AC_000179.1\|: 21770144 . . . 21770154 | 0.318 | 0.071 |
| ARS-USMARC-Parent-AY851162-no-rs | gi\|258513356\|ref\|AC_000168.1\|: 46411095 . . . 46411105 | 0.322 | 0.112 |
| ARS-USMARC-Parent-DQ916057- | gi\|258513360\|ref\|AC_000164.1\|: 81591567 . . . 81591607 | 0.323 | 0.157 |
| rs29009979 | | | |
| BTA-100621-no-rs | gi\|258513345\|ref\|AC_000179.1\|: 25598775 . . . 25598785 | 0.326 | 0.088 |
| ARS-BFGL-NGS-93119 | gi\|258513358\|ref\|AC_000166.1\|: 28149126 . . . 28149136 | 0.328 | 0.082 |
| ARS-USMARC-Parent-DQ984826- | gi\|258513353\|ref\|AC_000171.1\|: 27751883 . . . 27751893 | 0.328 | 0.063 |
| rs29027559 | | | |
| ARS-USMARC-Parent-AY853303-no-rs | gi\|258513354\|ref\|AC_000170.1\|: 75383369 . . . 75383379 | 0.328 | 0.071 |
| ARS-USMARC-Parent-DQ674265- | gi\|258513359\|ref\|AC_000165.1\|: 106174866 . . . 106174876 | 0.331 | 0.092 |
| rs29011266 | | | |
| ARS-USMARC-Parent-EF034087-no-rs | gi\|258513339\|ref\|AC_000185.1\|: 16097744 . . . 16097754 | 0.340 | 0.126 |
| ARS-USMARC-Parent-AY863214-rs17871744 | gi\|258513349\|ref\|AC_000175.1\|: 46647172 . . . 46647182 | 0.342 | 0.085 |
| ARS-USMARC-Parent-DQ837643- | gi\|258513356\|ref\|AC_000168.1\|: 66341584 . . . 66341594 | 0.343 | 0.092 |
| rs29018818 | | | |
| Hapmap39461-BTA-109898 | gi\|258513346\|ref\|AC_000178.1\|: 27927776 . . . 27927786 | 0.344 | 0.038 |
| ARS-USMARC-Parent-AY842474-rs29003226 | gi\|258513364\|ref\|AC_000160.1\|: 51976641 . . . 51976651 | 0.347 | 0.066 |
| ARS-USMARC-Parent-AY941204-rs17872131 | gi\|258513342\|ref\|AC_000182.1\|: 14683146 . . . 14683156 | 0.352 | 0.174 |
| ARS-USMARC-Parent-EF042091-rs29014974 | gi\|258513339\|ref\|AC_000185.1\|: 44261940 . . . 44261950 | 0.355 | 0.071 |
| ARS-USMARC-Parent-AY858890-rs29002256 | gi\|258513350\|ref\|AC_000174.1\|: 29936152 . . . 29936162 | 0.358 | 0.115 |
| ARS-USMARC-Parent-DQ500958-no-rs | gi\|258513362\|ref\|AC_000162.1\|: 27825113 . . . 27825123 | 0.358 | 0.156 |
| ARS-USMARC-569 | gi\|258513350\|ref\|AC_000174.1\|: 30360937 . . . 30360947 | 0.363 | 0.106 |
| ARS-BFGL-NGS-42505 | gi\|258513353\|ref\|AC_000171.1\|: 62478237 . . . 62478247 | 0.367 | 0.095 |
| ARS-USMARC-Parent-AY939849-rs17870274 | gi\|258513343\|ref\|AC_000181.1\|: 56415789 . . . 56415799 | 0.368 | 0.080 |
| ARS-BFGL-NGS-70946 | gi\|258513357\|ref\|AC_000167.1\|: 14574448 . . . 14574458 | 0.369 | 0.054 |
| ARS-USMARC-Parent-EF164803-rs29011141 | gi\|258513348\|ref\|AC_000176.1\|: 55174255 . . . 55174265 | 0.369 | 0.121 |
| ARS-USMARC-Parent-DQ786758- | gi\|258513360\|ref\|AC_000164.1\|: 18454631 . . . 18454641 | 0.369 | 0.106 |
| rs29024430 | | | |
| ARS-USMARC-Parent-EF028073-rs29014953 | gi\|258513349\|ref\|AC_000175.1\|: 1839728 . . . 1839738 | 0.370 | 0.183 |
| ARS-USMARC-Parent-AY916666-no-rs | gi\|258513348\|ref\|AC_000176.1\|: 44799385 . . . 44799395 | 0.378 | 0.094 |
| ARS-USMARC-Parent-AY914316-rs17871403 | gi\|258513349\|ref\|AC_000175.1\|: 48812009 . . . 48812019 | 0.381 | 0.087 |
| ARS-USMARC-Parent-EF034086-no-rs | gi\|258513341\|ref\|AC_000183.1\|: 38233332 . . . 38233342 | 0.382 | 0.263 |
| ARS-USMARC-Parent-DQ786766- | gi\|258513357\|ref\|AC_000167.1\|: 3530266 . . . 3530276 | 0.390 | 0.163 |
| rs29012070 | | | |
| ARS-USMARC-Parent-DQ650636- | gi\|258513359\|ref\|AC_000165.1\|: 28799244 . . . 28799254 | 0.393 | 0.212 |
| rs29024525 | | | |
| ARS-BFGL-NGS-67146 | gi\|258513365\|ref\|AC_000159.1\|: 118773628 . . . 118773638 | 0.393 | 0.099 |
| Hapmap24215-BTA-163266 | gi\|258513340\|ref\|AC_000184.1\|: 10764820 . . . 10764830 | 0.397 | 0.129 |
| ARS-BFGL-NGS-96125 | gi\|258513347\|ref\|AC_000177.1\|: 58449207 . . . 58449217 | 0.401 | 0.128 |
| ARS-BFGL-NGS-15506 | gi\|258513362\|ref\|AC_000162.1\|: 88334671 . . . 88334681 | 0.402 | 0.190 |
| ARS-BFGL-NGS-36513 | gi\|258513348\|ref\|AC_000176.1\|: 17236702 . . . 17236712 | 0.402 | 0.089 |
| ARS-USMARC-Parent-DQ647187- | gi\|258513364\|ref\|AC_000160.1\|: 21146872 . . . 21146882 | 0.403 | 0.099 |
| rs29010510 | | | |
| Hapmap31098-BTA-136127 | gi\|258513346\|ref\|AC_000178.1\|: 62028403 . . . 62028413 | 0.407 | 0.120 |
| ARS-USMARC-Parent-DQ866817-no-rs | gi\|258513352\|ref\|AC_000172.1\|: 38078770 . . . 38078780 | 0.413 | 0.221 |
| ARS-USMARC-Parent-DQ647186- | gi\|258513363\|ref\|AC_000161.1\|: 17200589 . . . 17200599 | 0.425 | 0.227 |
| rs29014143 | | | |
| Hapmap35535-SCAFFOLD86180_8791 | gi\|258513344\|ref\|AC_000180.1\|: 41700824 . . . 41700834 | 0.427 | 0.110 |
| ARS-USMARC-Parent-DQ404152- | gi\|258513365\|ref\|AC_000159.1\|: 5306833 . . . 5306843 | 0.429 | 0.285 |
| rs29022245 | | | |
| ARS-USMARC-Parent-DQ916058- | gi\|258513359\|ref\|AC_000165.1\|: 1554701 . . . 1554711 | 0.446 | 0.103 |
| rs29016146 | | | |
| ARS-BFGL-NGS-119662 | gi\|258513361\|ref\|AC_000163.1\|: 118292323 . . . 118292333 | 0.448 | 0.115 |
| ARS-USMARC-Parent-DQ984825- | gi\|258513357\|ref\|AC_000167.1\|: 98230474 . . . 98230484 | 0.465 | 0.072 |
| rs29012457 | | | |
| ARS-USMARC-Parent-EF026084-rs29025380 | gi\|258513348\|ref\|AC_000176.1\|: 15345307 . . . 15345317 | 0.468 | 0.182 |
| ARS-USMARC-Parent-DQ837645- | gi\|258513356\|ref\|AC_000168.1\|: 24553002 . . . 24553012 | 0.469 | 0.174 |
| rs29015870 | | | |
| ARS-USMARC-Parent-EF026086-rs29013660 | gi\|258513339\|ref\|AC_000185.1\|: 35331555 . . . 35331565 | 0.473 | 0.064 |
| BTB-00818821 | gi\|258513346\|ref\|AC_000178.1\|: 40408798 . . . 40408808 | 0.478 | 0.224 |
| ARS-BFGL-NGS-57711 | gi\|258513348\|ref\|AC_000176.1\|: 40692424 . . . 40692434 | 0.490 | 0.307 |
| ARS-USMARC-Parent-DQ846688- | gi\|258513362\|ref\|AC_000162.1\|: 119261604 . . . 119261614 | 0.495 | 0.134 |
| rs29023691 | | | |
| ARS-USMARC-Parent-DQ789028- | gi\|258513361\|ref\|AC_000163.1\|: 46936177 . . . 46936187 | 0.496 | 0.303 |
| rs29017713 | | | |
| ARS-USMARC-Parent-DQ470475-no-rs | gi\|258513362\|ref\|AC_000162.1\|: 7651048 . . . 7651058 | 0.513 | 0.115 |
| ARS-USMARC-Parent-AY937242-rs17872223 | gi\|258513344\|ref\|AC_000180.1\|: 27306790 . . . 27306800 | 0.519 | 0.190 |
| ARS-USMARC-Parent-EF034083-rs29018286 | gi\|258513342\|ref\|AC_000182.1\|: 3126433 . . . 3126443 | 0.522 | 0.110 |
| ARS-USMARC-Parent-AY842475-rs29002127 | gi\|258513363\|ref\|AC_000161.1\|: 20181744 . . . 20181754 | 0.524 | 0.285 |
| ARS-BFGL-NGS-55943 | gi\|258513352\|ref\|AC_000172.1\|: 64628701 . . . 64628711 | 0.557 | 0.108 |
| ARS-USMARC-Parent-DQ404150- | gi\|258517435\|ref\|AC_000158.1\|: 59409833 . . . 59409843 | 0.564 | 0.305 |
| rs29012530 | | | |
| ARS-USMARC-Parent-DQ404153-no-rs | gi\|258513338\|ref\|AC_000186.1\|: 44756497 . . . 44756507 | 0.567 | 0.163 |
| ARS-USMARC-Parent-EF150946-rs29023666 | gi\|258513341\|ref\|AC_000183.1\|: 13229214 . . . 13229224 | 0.567 | 0.166 |
| ARS-BFGL-NGS-102169 | gi\|258513340\|ref\|AC_000184.1\|: 12930431 . . . 12930441 | 0.579 | 0.241 |
| Hapmap42648-BTA-71195 | gi\|258513363\|ref\|AC_000161.1\|: 71061219 . . . 71061229 | 0.595 | 0.177 |
| ARS-USMARC-Parent-DQ435443- | gi\|258513364\|ref\|AC_000160.1\|: 58040465 . . . 58040475 | 0.600 | 0.258 |
| rs29010802 | | | |
| ARS-USMARC-Parent-DQ404151- | gi\|258517435\|ref\|AC_000158.1\|: 151349509 . . . 151349519 | 0.603 | 0.145 |
| rs29019282 | | | |
| ARS-USMARC-Parent-EF089234-rs29020870 | gi\|258513344\|ref\|AC_000180.1\|: 50884047 . . . 50884057 | 0.605 | 0.176 |
| ARS-USMARC-Parent-DQ846692- | gi\|258513353\|ref\|AC_000171.1\|: 80082918 . . . 80082928 | 0.623 | 0.125 |
| rs29010281 | | | |
| ARS-BFGL-NGS-112325 | gi\|258513355\|ref\|AC_000169.1\|: 79643098 . . . 79643108 | 0.624 | 0.266 |
| Hapmap41591-BTA-59790 | gi\|258513342\|ref\|AC_000182.1\|: 27912097 . . . 27912107 | 0.628 | 0.187 |
| ARS-BFGL-NGS-115514 | gi\|258513352\|ref\|AC_000172.1\|: 38003129 . . . 38003139 | 0.696 | 0.206 |
| ARS-USMARC-Parent-DQ990834- | gi\|258513341\|ref\|AC_000183.1\|: 8221265 . . . 8221275 | 0.708 | 0.229 |
| rs29013727 | | | |
| ARS-USMARC-Parent-EF042090-no-rs | gi\|258513352\|ref\|AC_000172.1\|: 21207524 . . . 21207534 | 0.714 | 0.283 |
| Hapmap43057-BTA-80741 | gi\|258513360\|ref\|AC_000164.1\|: 13279967 . . . 13279977 | 0.738 | 0.224 |
| ARS-USMARC-Parent-DQ916059- | gi\|258513349\|ref\|AC_000175.1\|: 23426209 . . . 23426219 | 0.745 | 0.143 |
| rs29009907 | | | |
| ARS-BFGL-NGS-117322 | gi\|258513356\|ref\|AC_000168.1\|: 77884739 . . . 77884749 | 0.753 | 0.098 |
| ARS-USMARC-Parent-AY856094-rs17871190 | gi\|258513338\|ref\|AC_000186.1\|: 9160934 . . . 9160944 | 0.759 | 0.198 |
| ARS-BFGL-NGS-42283 | gi\|258513356\|ref\|AC_000168.1\|: 95543406 . . . 95543416 | 0.759 | 0.185 |
| ARS-USMARC-Parent-DQ846690-no-rs | gi\|258513353\|ref\|AC_000171.1\|: 10171914 . . . 10171924 | 0.763 | 0.136 |
| ARS-BFGL-NGS-24419 | gi\|258513345\|ref\|AC_000179.1\|: 11756778 . . . 11756788 | 0.766 | 0.263 |
| ARS-BFGL-NGS-31640 | gi\|258513358\|ref\|AC_000166.1\|: 52430559 . . . 52430569 | 0.787 | 0.147 |
| ARS-USMARC-Parent-AY929334-no-rs | gi\|258513344\|ref\|AC_000180.1\|: 7219970 . . . 7219980 | 0.807 | 0.204 |
| ARS-USMARC-Parent-EF141102-rs29015783 | gi\|258513340\|ref\|AC_000184.1\|: 37513918 . . . 37513928 | 0.823 | 0.377 |
| ARS-BFGL-BAC-19454 | gi\|258513351\|ref\|AC_000173.1\|: 13695206 . . . 13695216 | 0.837 | 0.580 |
| ARS-USMARC-Parent-DQ489377- | gi\|258513364\|ref\|AC_000160.1\|: 98188379 . . . 98188389 | 1.093 | 0.264 |
| rs29026932 | | | |
| ARS-USMARC-Parent-DQ990833- | gi\|258513343\|ref\|AC_000181.1\|: 15447766 . . . 15447776 | 1.190 | 0.605 |
| rs29010147 | | | |
| Hapmap54547-rs29012198 | gi\|258513363\|ref\|AC_000161.1\|: 110776619 . . . 110776629 | 1.246 | 0.237 |
| Hapmap50598-BTA-122724 | gi\|258513348\|ref\|AC_000176.1\|: 4670551 . . . 4670561 | 1.248 | 0.502 |
| ARS-USMARC-Parent-AY851163-rs17871661 | gi\|258513356\|ref\|AC_000168.1\|: 103047469 . . . 103047479 | 1.358 | 0.437 |
| ARS-BFGL-NGS-86662 | gi\|258517435\|ref\|AC_000158.1\|: 40206012 . . . 40206022 | 1.373 | 0.349 |

What is claimed is:

1. A method for genotyping one or more target loci in a nucleic acid sample, comprising the steps of:
a) providing a nucleic acid sample;
b) adding a first set of primers to said sample to form a first amplification mixture, wherein the primers in said first set comprises a primer tail sequence and is capable of hybridizing to a target sequence within or adjacent to one or more of said target loci;
c) performing a first amplification reaction on said first amplification mixture to produce a first library of amplicons, wherein said amplicons comprise said primer tail sequence;
d) adding a second set of primers to said first library to form a second amplification mixture, wherein the primers in the second set are capable of hybridizing to said primer tail sequence; and
e) performing a second amplification reaction on said second amplification mixture to generate a second library of amplicons;
wherein for at least 90% of said target loci, the number of amplicons in said second library derived from each of said target loci deviates from the average number of amplicons for all target loci by less than one order of magnitude (+ or −10×).

2. The method of claim 1, wherein for at least 90% of said target loci, the number of amplicons in said second library derived from each of said target loci deviates from the average number of amplicons for all target loci by less than 5×.

3. The method of claim 2, wherein for at least 90% of said target loci, the number of amplicons in said second library derived from each of said target loci deviates from the average number of amplicons for all target loci by less than 2.5×.

4. The method of claim 1, wherein the number of target loci is greater than 10.

5. The method of claim 4, wherein the number of target loci is greater than 100.

6. The method of claim 1, wherein said first amplification reaction and said second amplification reaction are carried out simultaneously or consecutively.

7. The method of claim 6, wherein said first amplification reaction is carried out before said second amplification reaction.

8. The method of claim 7, further comprising purifying the first library after said first amplification reaction and before said second amplification reaction.

9. The method of claim 1, wherein said first amplification reaction comprises between 1 and 15 cycles.

10. The method of claim 9, wherein said first amplification reaction comprises between 2 and 10 cycles.

11. The method of claim 10, where said first amplification reaction comprises between 4 and 6 cycles.

12. The method of claim 1, wherein the primers in said first primer set are present in varying concentrations.

13. The method of claim 12, wherein the concentrations of said primers are calculated according to a regression equation.

14. The method of claim 1, wherein said second amplification reaction comprises at least one cycle.

15. The method of claim 14, wherein said second amplification reaction comprises between 5 and 50 cycles.

16. The method of claim 1, wherein said target loci are polymorphic genomic loci within a population.

17. The method of claim 1, further comprising the steps of:
f) obtaining sequence data from said first or said second library; and
h) determining the genotype at one or more of said target loci from said sequence data.

18. A method for identifying a novel polymorphic genomic locus in a sample, comprising the steps of:
a) providing two or more samples from individuals in a population;
b) subjecting each of said samples to the method of claim 17; and
c) aligning sequences corresponding to one or more target loci from two or more samples to identify target loci having sequence variation between individuals.

19. A kit for use in genotyping one or more target loci in a nucleic acid sample, comprising:
a) a first set of primers, wherein each primer in said first set comprises a primer tail sequence and is capable of hybridizing to a target sequence; and
b) a second set of primers, wherein each primer in said second set is capable of hybridizing to said primer tail sequence.


SEQUENCE LISTING (304 sequences)

SEQ ID NO: 1 - DNA, 35 bp, Artificial Sequence
  Primer
cacccacctgctctgacagacctttaattttggac

SEQ ID NO: 2 - DNA, 35 bp, Artificial Sequence
  Primer
ttacaatacaatacataaaaagctattcttaaatg

SEQ ID NO: 3 - DNA, 35 bp, Artificial Sequence
  Primer
cttcttggcttctgggcagttctgcaaacaggcaa

SEQ ID NO: 4 - DNA, 35 bp, Artificial Sequence
  Primer
cagcagagatctgaataggagtgttatttgcctca

SEQ ID NO: 5 - DNA, 35 bp, Artificial Sequence
  Primer
ccacttctctattctgactgcagccccatgaggta

SEQ ID NO: 6 - DNA, 35 bp, Artificial Sequence
  Primer
ataaattggaattaggtgaaaggaaaggaaagaag

SEQ ID NO: 7 - DNA, 35 bp, Artificial Sequence
  Primer
cctcaatgggaggcaaatccaaaacagagggcctg

SEQ ID NO: 8 - DNA, 35 bp, Artificial Sequence
  Primer
gagcactcgccacctgggtgtgcaaggtgccccga

SEQ ID NO: 9 - DNA, 35 bp, Artificial Sequence
  Primer
ctttgttgtctaatgtcagaaactataactatcta

SEQ ID NO: 10 - DNA, 35 bp, Artificial Sequence
  Primer
atctcctgtgtcctggctcaattgaagttacctgt

SEQ ID NO: 11 - DNA, 35 bp, Artificial Sequence
  Primer
tgtctaccatttatatgaaaagtattgtttaaact

SEQ ID NO: 12 - DNA, 35 bp, Artificial Sequence
  Primer
aaagccacacctattgctctatatgaacctgtgct

SEQ ID NO: 13 - DNA, 35 bp, Artificial Sequence
  Primer
ctgcagattctttacttgctgaggcattggagaag

SEQ ID NO: 14 - DNA, 35 bp, Artificial Sequence
  Primer
gccctccctcatgtcactgggacaccttggtacat

SEQ ID NO: 15 - DNA, 35 bp, Artificial Sequence
  Primer
atactcattacagtcaatctatttttacacagagg

SEQ ID NO: 16 - DNA, 35 bp, Artificial Sequence
  Primer
agactggtctgggatcccctcctccttatttccag

SEQ ID NO: 17 - DNA, 35 bp, Artificial Sequence
  Primer
ccatgcacctgatgggttgtttgtagcatccatta

SEQ ID NO: 18 - DNA, 35 bp, Artificial Sequence
  Primer
tcagggatgatttatttaaactctccaactaattt

SEQ ID NO: 19 - DNA, 35 bp, Artificial Sequence
  Primer
ctaaacatcttccaatgagtaggacaggccctgtg

SEQ ID NO: 20 - DNA, 35 bp, Artificial Sequence
  Primer
attgaaccgtgttctctagcactacagtcagagtc

SEQ ID NO: 21 - DNA, 35 bp, Artificial Sequence
  Primer
aaccggggagcctttggagtgagtggcctgagagg

SEQ ID NO: 22 - DNA, 35 bp, Artificial Sequence
  Primer
attctctcctctcattataatcctgcatggtggtg

SEQ ID NO: 23 - DNA, 35 bp, Artificial Sequence
  Primer
actcttctgtccacaaggtctggctccatcctggt

SEQ ID NO: 24 - DNA, 35 bp, Artificial Sequence
  Primer
tgacttccattaaagttctcctgttttgaccatca

SEQ ID NO: 25 - DNA, 35 bp, Artificial Sequence
  Primer
atcagcccacatatcatgcaattcacactcacata

SEQ ID NO: 26 - DNA, 35 bp, Artificial Sequence
  Primer
tgcttttattattaatttactatcttagacaaaca

SEQ ID NO: 27 - DNA, 35 bp, Artificial Sequence
  Primer
tttaaaaatgcaggccaatcagtgaaaaatacacc

SEQ ID NO: 28 - DNA, 35 bp, Artificial Sequence
  Primer
ttttcgcttctgttcttttcacagctatttgtaag

SEQ ID NO: 29 - DNA, 35 bp, Artificial Sequence
  Primer
acaggatgaatactaacatggccctcctttctctt

SEQ ID NO: 30 - DNA, 35 bp, Artificial Sequence
  Primer
ctggggaaagtttggggcacttgtaaaatgcttgc

SEQ ID NO: 31 - DNA, 35 bp, Artificial Sequence
  Primer
ataaaccacacgctcaggagtgaaccgtgccaaag

SEQ ID NO: 32 - DNA, 35 bp, Artificial Sequence
  Primer
gtatttctcgcatgacatttgcagcctaatgtaga

SEQ ID NO: 33 - DNA, 35 bp, Artificial Sequence
  Primer
gcatagaactccttgaaaatgggatggagtaatct

SEQ ID NO: 34 - DNA, 34 bp, Artificial Sequence
  Primer
tttaaagatttctttccaaagtgcccccatctac

SEQ ID NO: 35 - DNA, 35 bp, Artificial Sequence
  Primer
aggacccaagtacgaaagggttagtgatgttccaa

SEQ ID NO: 36 - DNA, 35 bp, Artificial Sequence
  Primer
ctcagttcctaggtggtgctgacatactggtggtc

SEQ ID NO: 37 - DNA, 35 bp, Artificial Sequence
  Primer
catctttatttggttaggaaactctcctccgtcag

SEQ ID NO: 38 - DNA, 35 bp, Artificial Sequence
  Primer
atctttacccagcttcgcccaagcagccaatagca

SEQ ID NO: 39 - DNA, 35 bp, Artificial Sequence
  Primer
aaatttacaaaaaagtaagaagctagtagagagaa

SEQ ID NO: 40 - DNA, 35 bp, Artificial Sequence
  Primer
tgtcccagtccaaaggagcctcaggggatatgaca

SEQ ID NO: 41 - DNA, 35 bp, Artificial Sequence
  Primer
actttccttgtctggcttccttttcttcttttttt

SEQ ID NO: 42 - DNA, 35 bp, Artificial Sequence
  Primer
tgtgtgtgagacaagccgttccatagccatccaga

SEQ ID NO: 43 - DNA, 35 bp, Artificial Sequence
  Primer
agccaagtgaggtgatacttactggttcagaacga

SEQ ID NO: 44 - DNA, 35 bp, Artificial Sequence
  Primer
tggaagacagggtataagaaaggaagggtgcctga

SEQ ID NO: 45 - DNA, 35 bp, Artificial Sequence
  Primer
taagccccctactgtgaaacgtggaaggagtcagc

SEQ ID NO: 46 - DNA, 35 bp, Artificial Sequence
  Primer
gggaggccaaagggaatctcaggtggagacatcag

SEQ ID NO: 47 - DNA, 35 bp, Artificial Sequence
  Primer
gaagtatattcctgcaataaataaatctcaggata

SEQ ID NO: 48 - DNA, 35 bp, Artificial Sequence
  Primer
gaaggctgctctgatgattcctgctggaaagcaag

SEQ ID NO: 49 - DNA, 35 bp, Artificial Sequence
  Primer
tttttttgtctccatgtttttttattttaccttca

SEQ ID NO: 50 - DNA, 35 bp, Artificial Sequence
  Primer
actggcaagtttactcttgttgctgttttaaatgc

SEQ ID NO: 51 - DNA, 35 bp, Artificial Sequence
  Primer
caatcttacctgtgcctccctgctccttgccccca

SEQ ID NO: 52 - DNA, 35 bp, Artificial Sequence
  Primer
tggagatggggttcagtgctctgaaggaactgagg

SEQ ID NO: 53 - DNA, 35 bp, Artificial Sequence
  Primer
aactgcctaatggcctgatactggcctgtggcctg

SEQ ID NO: 54 - DNA, 35 bp, Artificial Sequence
  Primer
ggagacccacgtggcaaggagcttgggtagtctct

SEQ ID NO: 55 - DNA, 35 bp, Artificial Sequence
  Primer
atgctttctaaatccaagatctagactccattatt

SEQ ID NO: 56 - DNA, 35 bp, Artificial Sequence
  Primer
atccagggatagaacctgtgtctctcaagtctcct

SEQ ID NO: 57 - DNA, 35 bp, Artificial Sequence
  Primer
aaacacacagtcaggggaggccatggaaaggagag

SEQ ID NO: 58 - DNA, 35 bp, Artificial Sequence
  Primer
gctgggagaggtagattgagatttgggacaagagt

SEQ ID NO: 59 - DNA, 35 bp, Artificial Sequence
  Primer
tacttttccttcctttgagtctgaatcctttaaca

SEQ ID NO: 60 - DNA, 35 bp, Artificial Sequence
  Primer
tgattgctctcttctggaagttctccagacagttt

SEQ ID NO: 61 - DNA, 35 bp, Artificial Sequence
  Primer
ggctcctggcagagttcgggtttagtcaccatgcc

SEQ ID NO: 62 - DNA, 35 bp, Artificial Sequence
  Primer
catgtaatgttcacagatggcccaaccaagtgtct

SEQ ID NO: 63 - DNA, 35 bp, Artificial Sequence
  Primer
cccaacactggggattgaggagagctccagattcc

SEQ ID NO: 64 - DNA, 35 bp, Artificial Sequence
  Primer
gtggggcaggaaaggaggtaggctcgtagaatgtg

SEQ ID NO: 65 - DNA, 35 bp, Artificial Sequence
  Primer
ttgtttttccttctgtagatgttaactggtaacca

SEQ ID NO: 66 - DNA, 35 bp, Artificial Sequence
  Primer
caaacctcaattttgtctgttttggcatctcccca

SEQ ID NO: 67 - DNA, 35 bp, Artificial Sequence
  Primer
ttaagaggaggcggtgctcaccccactgctgaagg

SEQ ID NO: 68 - DNA, 35 bp, Artificial Sequence
  Primer
aatcgcgtccacacccaggtcggaggcacagaaag

SEQ ID NO: 69 - DNA, 35 bp, Artificial Sequence
  Primer
agggttggcaagcttaggaggaaaccagtcatctc

SEQ ID NO: 70 - DNA, 35 bp, Artificial Sequence
  Primer
ctatttctatatatacatttttcctttccatgcta

SEQ ID NO: 71 - DNA, 35 bp, Artificial Sequence
  Primer
aacttcaatttaatgagatttcaatgtgcctttat

SEQ ID NO: 72 - DNA, 35 bp, Artificial Sequence
  Primer
ataaaaaggaatgaagttctgacaatgctacaagt

SEQ ID NO: 73 - DNA, 35 bp, Artificial Sequence
  Primer
ctcagtcacagtctcagctcccaactcagttttac

SEQ ID NO: 74 - DNA, 35 bp, Artificial Sequence
  Primer
ttgggtcttggtgtttgttactacattttagtgtt

SEQ ID NO: 75 - DNA, 35 bp, Artificial Sequence
  Primer
atatgcacacatgtatcacttatccaaatatttaa

SEQ ID NO: 76 - DNA, 35 bp, Artificial Sequence
  Primer
tggccttggggccagaaaggatgtactaagacagg

SEQ ID NO: 77 - DNA, 35 bp, Artificial Sequence
  Primer
ttcaaggttgtatgctagtttgtgaactgagttca

SEQ ID NO: 78 - DNA, 35 bp, Artificial Sequence
  Primer
tgtgtcacaaaacatttttcttctcccggttattt

SEQ ID NO: 79 - DNA, 35 bp, Artificial Sequence
  Primer
cttataagcatcaatcagaaaatgctgggttgaca

SEQ ID NO: 80 - DNA, 35 bp, Artificial Sequence
  Primer
gagtatagttgctttacaatgttgtgttagcttat

SEQ ID NO: 81 - DNA, 35 bp, Artificial Sequence
  Primer
ccagaccatggaaataatttgactatatgccacaa

SEQ ID NO: 82 - DNA, 35 bp, Artificial Sequence
  Primer
ttgcggaaaagatagaaccatttatttttgttttg

SEQ ID NO: 83 - DNA, 35 bp, Artificial Sequence
  Primer
aagcctcttgaaagcaagccagagccagcagcctc

SEQ ID NO: 84 - DNA, 35 bp, Artificial Sequence
  Primer
attttctggctttccccctgagtgccttctctctc

SEQ ID NO: 85 - DNA, 35 bp, Artificial Sequence
  Primer
tatgagcacctccggggcgtctgtccctgaaataa

SEQ ID NO: 86 - DNA, 35 bp, Artificial Sequence
  Primer
atatgaagcacattttgaaactagcctatgcctct

SEQ ID NO: 87 - DNA, 35 bp, Artificial Sequence
  Primer
aaaacagaaaactacaggtagtttgtcttatatta

SEQ ID NO: 88 - DNA, 35 bp, Artificial Sequence
  Primer
attattggtaggtttagctggaatctggtatctgg

SEQ ID NO: 89 - DNA, 35 bp, Artificial Sequence
  Primer
tgagtccccacattattttagctgttttagcaaac

SEQ ID NO: 90 - DNA, 35 bp, Artificial Sequence
  Primer
ccggccttgagcacctgcagcggtcactcatggaa

SEQ ID NO: 91 - DNA, 35 bp, Artificial Sequence
  Primer
gcgatttcatttggtatcacatccttgctgggctt

SEQ ID NO: 92 - DNA, 35 bp, Artificial Sequence
  Primer
gaatcagacacaattgagtgactttcactttctga

SEQ ID NO: 93 - DNA, 35 bp, Artificial Sequence
  Primer
tctccaagctcccaagggcatctctctacgtaata

SEQ ID NO: 94 - DNA, 35 bp, Artificial Sequence
  Primer
tattaagtagcctgtcctttccatacccctgccac

SEQ ID NO: 95 - DNA, 35 bp, Artificial Sequence
  Primer
tagagacatcaggccagcacaagccacggagtagc

SEQ ID NO: 96 - DNA, 35 bp, Artificial Sequence
  Primer
gaaagggaaaagtgaaagtgaagttgctcagtctt

SEQ ID NO: 97 - DNA, 35 bp, Artificial Sequence
  Primer
ttccttgtggcctgcttctcacaaagacacttgac

SEQ ID NO: 98 - DNA, 35 bp, Artificial Sequence
  Primer
cagtatcctataagccaagcagccatcccatcaac

SEQ ID NO: 99 - DNA, 35 bp, Artificial Sequence
  Primer
gcagggtctgctcaccacaacacagtcacacctcc

SEQ ID NO: 100 - DNA, 35 bp, Artificial Sequence
  Primer
aacagttatctactgtggtcttggaggtctgtgtt

SEQ ID NO: 101 - DNA, 35 bp, Artificial Sequence
  Primer
tccctgcacacaacctgaagttagctccagggcag

SEQ ID NO: 102 - DNA, 35 bp, Artificial Sequence
  Primer
caagctggggcttcttctatgttcagtggctgtca

SEQ ID NO: 103 - DNA, 35 bp, Artificial Sequence
  Primer
cctatcattccacagagttgtataaattcccctgg

SEQ ID NO: 104 - DNA, 35 bp, Artificial Sequence
  Primer
gttgactcctcctactcctccatctccttttcatt

SEQ ID NO: 105 - DNA, 35 bp, Artificial Sequence
  Primer
tactattatctgggtacttctgagagcatgtggga

SEQ ID NO: 106 - DNA, 35 bp, Artificial Sequence
  Primer
gttctcatggtctaaaccagaggaaaggcaggcta

SEQ ID NO: 107 - DNA, 35 bp, Artificial Sequence
  Primer
ctgagctatgagggaagtccatataaaccatgtac

SEQ ID NO: 108 - DNA, 35 bp, Artificial Sequence
  Primer
tattttctagtgggccaatttaattcccatggtgt

SEQ ID NO: 109 - DNA, 35 bp, Artificial Sequence
  Primer
tcatagtgaaaggtgtcacaactttgcatcataaa

SEQ ID NO: 110 - DNA, 35 bp, Artificial Sequence
  Primer
cctctggagaaaagagctgtatttgctgtccccat

SEQ ID NO: 111 - DNA, 35 bp, Artificial Sequence
  Primer
actacacgccctcacagttgattaatgatagcaga

SEQ ID NO: 112 - DNA, 35 bp, Artificial Sequence
  Primer
ggcatggcattttcagagggccttaatgcgaagtg

SEQ ID NO: 113 - DNA, 35 bp, Artificial Sequence
  Primer
gctttagaacatctacaggcctgggtgagggctac

SEQ ID NO: 114 - DNA, 35 bp, Artificial Sequence
  Primer
cttcagcctttagaaccattaaggatcaatgaaca

SEQ ID NO: 115 - DNA, 35 bp, Artificial Sequence
  Primer
tgtaatgagtaagcactggctgaatgggtgaatct

SEQ ID NO: 116 - DNA, 35 bp, Artificial Sequence
  Primer
gctgaccttttccctaggacctcatctcacacact

SEQ ID NO: 117 - DNA, 35 bp, Artificial Sequence
  Primer
cgggacactgggcctccgtaaaaacctgggggatc

SEQ ID NO: 118 - DNA, 35 bp, Artificial Sequence
  Primer
atcttccaaaggttcggaatctcggagtctcagtt

SEQ ID NO: 119 - DNA, 35 bp, Artificial Sequence
  Primer
tttgttatttttggttgcagtggatctttgtccct

SEQ ID NO: 120 - DNA, 35 bp, Artificial Sequence
  Primer
agggacttccctgatggtatgatagataagaatcc

SEQ ID NO: 121 - DNA, 35 bp, Artificial Sequence
  Primer
ttctttttctttatttttgaatgtgctgggtcttc

SEQ ID NO: 122 - DNA, 35 bp, Artificial Sequence
  Primer
tggtccaggaagattcaacatgccacaggacaata

SEQ ID NO: 123 - DNA, 35 bp, Artificial Sequence
  Primer
gatggttttaacacacggtgcagaatgtagactca

SEQ ID NO: 124 - DNA, 35 bp, Artificial Sequence
  Primer
cagagtgtactccttgcaaaatattgtattattag

SEQ ID NO: 125 - DNA, 35 bp, Artificial Sequence
  Primer
atcggcagaaacatatttgagaccctggccatcgt

SEQ ID NO: 126 - DNA, 35 bp, Artificial Sequence
  Primer
aatcaaaagctaggcagtgagggagttggcaggga

SEQ ID NO: 127 - DNA, 35 bp, Artificial Sequence
  Primer
tcatgtcccctcacatagtttttaaggttatttat

SEQ ID NO: 128 - DNA, 35 bp, Artificial Sequence
  Primer
tatacaccaaaaataaaattcctacaatgacctac

SEQ ID NO: 129 - DNA, 35 bp, Artificial Sequence
  Primer
agatgtgctgcatcgtgacggcttccaggatctga

SEQ ID NO: 130 - DNA, 35 bp, Artificial Sequence
  Primer
cacatgattgaaaaagctgagttaacaggaacacc

SEQ ID NO: 131 - DNA, 35 bp, Artificial Sequence
  Primer
ctgcagctacattcttcccttcccttctgccagtc

SEQ ID NO: 132 - DNA, 35 bp, Artificial Sequence
  Primer
tcttctacaagacagaaatcaagaatacagttgaa

SEQ ID NO: 133 - DNA, 35 bp, Artificial Sequence
  Primer
catagttttcttccacagtcaacccaactgtgagg

SEQ ID NO: 134 - DNA, 35 bp, Artificial Sequence
  Primer
attgtaacagagtttaagaaaaacgaaagcataat

SEQ ID NO: 135 - DNA, 35 bp, Artificial Sequence
  Primer
ttcagtagtgtccacactttgtgaacccttggact

SEQ ID NO: 136 - DNA, 35 bp, Artificial Sequence
  Primer
taggggcttccccagtggctcagcggtaaagaatc

SEQ ID NO: 137 - DNA, 35 bp, Artificial Sequence
  Primer
ttcacaggctctggcaatgtggccttgaggactta

SEQ ID NO: 138 - DNA, 35 bp, Artificial Sequence
  Primer
ggttcacgtcctggcttgcgaagatcctctggagt

SEQ ID NO: 139 - DNA, 35 bp, Artificial Sequence
  Primer
cagagctggtaggtagccttttggttcacttagca

SEQ ID NO: 140 - DNA, 35 bp, Artificial Sequence
  Primer
gcagtagtgaatttatggtagactgaatgcctgcc

SEQ ID NO: 141 - DNA, 35 bp, Artificial Sequence
  Primer
cctggggaactgtggacctgtgggcaatcccttag

SEQ ID NO: 142 - DNA, 35 bp, Artificial Sequence
  Primer
cactccagcttggagcttaaccaagtctttactgt

SEQ ID NO: 143 - DNA, 35 bp, Artificial Sequence
  Primer
taaagctactgagctgttcacaagtagtctaactt

SEQ ID NO: 144 - DNA, 35 bp, Artificial Sequence
  Primer
cccccagctccctgcttagacatcacttcctccag

SEQ ID NO: 145 - DNA, 35 bp, Artificial Sequence
  Primer
tcactcaattagttaacactctggacacagcattg

SEQ ID NO: 146 - DNA, 35 bp, Artificial Sequence
  Primer
tattttcggtatccccatatcatgactaaatacac

SEQ ID NO: 147 - DNA, 35 bp, Artificial Sequence
  Primer
ctcctccttcagcaagactcaggcccttcctctct

SEQ ID NO: 148 - DNA, 35 bp, Artificial Sequence
  Primer
atgaacttggagcgggtgtccaacgaagagaagtt

SEQ ID NO: 149 - DNA, 35 bp, Artificial Sequence
  Primer
gctgtataattcatcagccctcacccctcccagtc

SEQ ID NO: 150 - DNA, 35 bp, Artificial Sequence
  Primer
accaagagccgtgcctctgcacagtagatgtggat

SEQ ID NO: 151 - DNA, 35 bp, Artificial Sequence
  Primer
tttcaattgctgccagactgaaaaggcagagaacc

SEQ ID NO: 152 - DNA, 35 bp, Artificial Sequence
  Primer
ttggctggacagttcatgtttaactcttgttaagg

SEQ ID NO: 153 - DNA, 35 bp, Artificial Sequence
  Primer
cactgcacatggatattgtctctccctcctctaaa

SEQ ID NO: 154 - DNA, 35 bp, Artificial Sequence
  Primer
actccagtcaggatccatatcgctgtctacctagt

SEQ ID NO: 155 - DNA, 35 bp, Artificial Sequence
  Primer
tctcatgccaagctaagagcagtttcacgtagtat

SEQ ID NO: 156 - DNA, 35 bp, Artificial Sequence
  Primer
ttacttgattgtcactgtcacatccatcattttgt

SEQ ID NO: 157 - DNA, 35 bp, Artificial Sequence
  Primer
ctatgatgaagcaataaggcactgatgaattgtgg

SEQ ID NO: 158 - DNA, 35 bp, Artificial Sequence
  Primer
ctggctctgtttcttttctttggcttttcatcttt

SEQ ID NO: 159 - DNA, 35 bp, Artificial Sequence
  Primer
ttataataaccagtgttggtaaggacgtagaaaag

SEQ ID NO: 160 - DNA, 35 bp, Artificial Sequence
  Primer
acaaacaacagtttaattgataatatccttacaga

SEQ ID NO: 161 - DNA, 35 bp, Artificial Sequence
  Primer
aatgctttttcagcatccctccccactctgctaat

SEQ ID NO: 162 - DNA, 35 bp, Artificial Sequence
  Primer
cacatctcagagacctcggtcatcgtcaagatgga

SEQ ID NO: 163 - DNA, 35 bp, Artificial Sequence
  Primer
cagccacctctgtactggtcgcatcttagaggaaa

SEQ ID NO: 164 - DNA, 35 bp, Artificial Sequence
  Primer
ttggcctgcctgccgaggagtaagttgggattttt

SEQ ID NO: 165 - DNA, 35 bp, Artificial Sequence
  Primer
ccagcatcctttcagcagaagcccaagctcaccag

SEQ ID NO: 166 - DNA, 35 bp, Artificial Sequence
  Primer
actgactttggggcctgatgtgggctcaagaactg

SEQ ID NO: 167 - DNA, 35 bp, Artificial Sequence
  Primer
cagaactacactaccccacttcctctgtgacaaca

SEQ ID NO: 168 - DNA, 35 bp, Artificial Sequence
  Primer
aagcctgggttgagccttcactattcttaagtcct

SEQ ID NO: 169 - DNA, 35 bp, Artificial Sequence
  Primer
actaatcacagcacaggtgacctcagaattcacta

SEQ ID NO: 170 - DNA, 35 bp, Artificial Sequence
  Primer
taccatctttggtttcctactttcttccatcctga

SEQ ID NO: 171 - DNA, 35 bp, Artificial Sequence
  Primer
ctttctcatgttattccgtggggttatctctgaga

SEQ ID NO: 172 - DNA, 35 bp, Artificial Sequence
  Primer
caataggtgaacgaatgaatgaataagtgaatgac

SEQ ID NO: 173 - DNA, 35 bp, Artificial Sequence
  Primer
tgagttgggaatgcagtttttctcctctaatgtta

SEQ ID NO: 174 - DNA, 35 bp, Artificial Sequence
  Primer
actctgcgacatgtgggatcttcttggaccagggg

SEQ ID NO: 175 - DNA, 35 bp, Artificial Sequence
  Primer
gttgtctgataagacattgacaggggcagggtaga

SEQ ID NO: 176 - DNA, 35 bp, Artificial Sequence
  Primer
ggtcatgaacattaattctagcataaaactcatgt

SEQ ID NO: 177 - DNA, 35 bp, Artificial Sequence
  Primer
tgcctggatctgagaggtgggacaaggaggtggct

SEQ ID NO: 178 - DNA, 35 bp, Artificial Sequence
  Primer
taccagcacccaccacattggaatctctctctcct

SEQ ID NO: 179 - DNA, 35 bp, Artificial Sequence
  Primer
gggagaggagagctcttctcagattgcctgatttc

SEQ ID NO: 180 - DNA, 35 bp, Artificial Sequence
  Primer
ctcaaggaatcagagatgggtgtgaccgattggat

SEQ ID NO: 181 - DNA, 35 bp, Artificial Sequence
  Primer
tgcaaataatctcttatttactctcagagtatcca

SEQ ID NO: 182 - DNA, 35 bp, Artificial Sequence
  Primer
gggttcagtgggcagaatcctataatgatcctctt

SEQ ID NO: 183 - DNA, 35 bp, Artificial Sequence
  Primer
ctgcaaattgctcatcatggatcctgtgatctatc

SEQ ID NO: 184 - DNA, 35 bp, Artificial Sequence
  Primer
aagaccagagatgacagtaggtgaggcaagaaagg

SEQ ID NO: 185 - DNA, 35 bp, Artificial Sequence
  Primer
cacctgggtcatggaatggaaaattatcaaagtct

SEQ ID NO: 186 - DNA, 35 bp, Artificial Sequence
  Primer
taagttcatatgacaataggatgaactcatgaaca

SEQ ID NO: 187 - DNA, 35 bp, Artificial Sequence
  Primer
taccctggaaaagaaatcctccctttctcccctcc

SEQ ID NO: 188 - DNA, 35 bp, Artificial Sequence
  Primer
tcccgctcctttcccgccactcggcgttcctaccc

SEQ ID NO: 189 - DNA, 35 bp, Artificial Sequence
  Primer
tgggaagtagcactggggatggttaaggattcagc

SEQ ID NO: 190 - DNA, 35 bp, Artificial Sequence
  Primer
ttctgatttttaaagcatgacctcattccaacaca

SEQ ID NO: 191 - DNA, 35 bp, Artificial Sequence
  Primer
ccatcccccaggcccagcctcttctctgtgagccc

SEQ ID NO: 192 - DNA, 35 bp, Artificial Sequence
  Primer
ttagcacgcatgcaaagagtgattaaaatgctaaa

SEQ ID NO: 193 - DNA, 35 bp, Artificial Sequence
  Primer
gttgtgaagagccagacatgactgagcaactgaac

SEQ ID NO: 194 - DNA, 35 bp, Artificial Sequence
  Primer
ttgctgaattccttgatgagttctattaattggtg

SEQ ID NO: 195 - DNA, 35 bp, Artificial Sequence
  Primer
ttcgttcgcaatgtcctttctctctttgttttgct

SEQ ID NO: 196 - DNA, 35 bp, Artificial Sequence
  Primer
atcgaaccctcatctcttatgtctcctgcactgac

SEQ ID NO: 197 - DNA, 35 bp, Artificial Sequence
  Primer
gaaccttgggttttcctggatacagggctcagtgg

SEQ ID NO: 198 - DNA, 35 bp, Artificial Sequence
  Primer
aggagggctgctttcttgtgctgagtagactttca

SEQ ID NO: 199 - DNA, 35 bp, Artificial Sequence
  Primer
tgcagtgattatcagccgaaaatcaccccctttgc

SEQ ID NO: 200 - DNA, 35 bp, Artificial Sequence
  Primer
ctactgtccagctgcagccacactgcccagtccta

SEQ ID NO: 201 - DNA, 35 bp, Artificial Sequence
  Primer
ttaaccctgactaaataggtttgggacaatctact

SEQ ID NO: 202 - DNA, 35 bp, Artificial Sequence
  Primer
catggggtcataggaaaaatatctcacatttcagc

SEQ ID NO: 203 - DNA, 35 bp, Artificial Sequence
  Primer
agacagggagaagggaaagtttatgagaaaagaca

SEQ ID NO: 204 - DNA, 35 bp, Artificial Sequence
  Primer
cgtgtctgactctttgtgatgctgtatgctgtcac

SEQ ID NO: 205 - DNA, 35 bp, Artificial Sequence
  Primer
ggggtctcagtttactctgccgcaaggcactgctc

SEQ ID NO: 206 - DNA, 35 bp, Artificial Sequence
  Primer
tttcaatgcagggggtgaaggttcaatccatggtc

SEQ ID NO: 207 - DNA, 35 bp, Artificial Sequence
  Primer
gcccaggatgtgccctatgaccctaggagaggggg

SEQ ID NO: 208 - DNA, 35 bp, Artificial Sequence
  Primer
aagcccaagaataccagagtgtatagcctatccct

SEQ ID NO: 209 - DNA, 35 bp, Artificial Sequence
  Primer
tggagcaccagggcattacctatcacagaatttta

SEQ ID NO: 210 - DNA, 35 bp, Artificial Sequence
  Primer
aactctctcgggtgcttctcagaaagccttcttct

SEQ ID NO: 211 - DNA, 35 bp, Artificial Sequence
  Primer
gactgagaggccgtttgggtttttgcaccaacgtg

SEQ ID NO: 212 - DNA, 35 bp, Artificial Sequence
  Primer
cagaattcgaacggttccgggattcaggacagaag

SEQ ID NO: 213 - DNA, 35 bp, Artificial Sequence
  Primer
tcaagtgttcttctctgaatggcttttaaaggctc

SEQ ID NO: 214 - DNA, 35 bp, Artificial Sequence
  Primer
gtaaacatcttccagtaagtgctccggactcccaa

SEQ ID NO: 215 - DNA, 35 bp, Artificial Sequence
  Primer
tagaacaactggctcatcaacagctattaattatt

SEQ ID NO: 216 - DNA, 35 bp, Artificial Sequence
  Primer
ccgaaggacagactggactggcagctacagagtcg

SEQ ID NO: 217 - DNA, 35 bp, Artificial Sequence
  Primer
gacagacaacaggtggcagctcggcagcagtcggc

SEQ ID NO: 218 - DNA, 35 bp, Artificial Sequence
  Primer
ggggctggaatctgttgacaagactttcctgaggc

SEQ ID NO: 219 - DNA, 35 bp, Artificial Sequence
  Primer
gatcaaggctacaaaccctgagatggggaacatac

SEQ ID NO: 220 - DNA, 35 bp, Artificial Sequence
  Primer
tacttccttgtcttttccagcttctagagaccatc

SEQ ID NO: 221 - DNA, 35 bp, Artificial Sequence
  Primer
ttcagaagtaagataaaattgtgaaaataaacacc

SEQ ID NO: 222 - DNA, 35 bp, Artificial Sequence
  Primer
ggagggaatttaagaaacagctaaatgtggcaact

SEQ ID NO: 223 - DNA, 35 bp, Artificial Sequence
  Primer
ccaccaatgctcacttctgggaacaggccctgcat

SEQ ID NO: 224 - DNA, 35 bp, Artificial Sequence
  Primer
ccctccttattctccatgagatcatttcaacaaac

SEQ ID NO: 225 - DNA, 35 bp, Artificial Sequence
  Primer
tgcctagggcggggctacaccaggatccgaggtca

SEQ ID NO: 226 - DNA, 35 bp, Artificial Sequence
  Primer
ccagacaaggctgggaacagaaccacttcaaggac

SEQ ID NO: 227 - DNA, 35 bp, Artificial Sequence
  Primer
tttcaaagcaacacatctggtcatcactcaccaac

SEQ ID NO: 228 - DNA, 35 bp, Artificial Sequence
  Primer
gtcgagaaaccaaacccagagtggaacttcaaaac

SEQ ID NO: 229 - DNA, 35 bp, Artificial Sequence
  Primer
catttctctgtggaaacataagcgggaagcatgaa

SEQ ID NO: 230 - DNA, 35 bp, Artificial Sequence
  Primer
ctacataagctagaatttaaaacacagtgtcaact

SEQ ID NO: 231 - DNA, 35 bp, Artificial Sequence
  Primer
gaacaaaacagagcaaaaatccttgccttcatggg

SEQ ID NO: 232 - DNA, 35 bp, Artificial Sequence
  Primer
ctccatccagccacacaaactcctcattcattgaa

SEQ ID NO: 233 - DNA, 35 bp, Artificial Sequence
  Primer
tcttgttttctccccatagaacttggcacattttg

SEQ ID NO: 234 - DNA, 35 bp, Artificial Sequence
  Primer
taattacttataagaggtaagctctgtctctcaag

SEQ ID NO: 235 - DNA, 35 bp, Artificial Sequence
  Primer
gtttttaaaagcttgcatctctaagctgtatgtgt

SEQ ID NO: 236 - DNA, 35 bp, Artificial Sequence
  Primer
ctatcagtccttgaatatcacagaagatagggaac

SEQ ID NO: 237 - DNA, 35 bp, Artificial Sequence
  Primer
cacacacatacacacacatacacacatcactactt

SEQ ID NO: 238 - DNA, 35 bp, Artificial Sequence
  Primer
agcaatttcattatggggccctgagccaaagatgc

SEQ ID NO: 239 - DNA, 35 bp, Artificial Sequence
  Primer
cactttcactcaaaggcataggagacaattaggaa

SEQ ID NO: 240 - DNA, 35 bp, Artificial Sequence
  Primer
gaaatgagacctagcatgatgaccaccaagtcaca

SEQ ID NO: 241 - DNA, 35 bp, Artificial Sequence
  Primer
gccagttttcctgcagttctctcctgtctggctca

SEQ ID NO: 242 - DNA, 35 bp, Artificial Sequence
  Primer
aggccgcctcttcctcttcactctcctcctcgctc

SEQ ID NO: 243 - DNA, 35 bp, Artificial Sequence
  Primer
ggcaccaagaaaaaaggcaggaagtggcaataacc

SEQ ID NO: 244 - DNA, 35 bp, Artificial Sequence
  Primer
ggttcttgtctctcctcacctgggctataacaatg

SEQ ID NO: 245 - DNA, 35 bp, Artificial Sequence
  Primer
agggagcacgcggaacagtgtttccagaagtagca

SEQ ID NO: 246 - DNA, 35 bp, Artificial Sequence
  Primer
ctctcttcccagacatctcaaaaccggatctccat

SEQ ID NO: 247 - DNA, 35 bp, Artificial Sequence
  Primer
taacagagacctcagaaaggatgtgtgttgtccac

SEQ ID NO: 248 - DNA, 35 bp, Artificial Sequence
  Primer
tccccatgtggacttgtagaacgctgtgtgtgacc

SEQ ID NO: 249 - DNA, 35 bp, Artificial Sequence
  Primer
tgaaggttgtagcagaaggcgaaataattcataat

SEQ ID NO: 250 - DNA, 35 bp, Artificial Sequence
  Primer
tctgtttctttggtgaagagtacctgtttccttcc

SEQ ID NO: 251 - DNA, 35 bp, Artificial Sequence
  Primer
tgatactgctgaattactgctcccatgatacgcac

SEQ ID NO: 252 - DNA, 35 bp, Artificial Sequence
  Primer
gactgtggcctgatcagactgtgtcctctgctgcc

SEQ ID NO: 253 - DNA, 35 bp, Artificial Sequence
  Primer
gccctcccccacctcccttcccctgcagccttgac

SEQ ID NO: 254 - DNA, 35 bp, Artificial Sequence
  Primer
agcgccgcttcagttcctgggggaacttggtggtt

SEQ ID NO: 255 - DNA, 35 bp, Artificial Sequence
  Primer
catagtcagcactttgtaagcacccactaacttgc

SEQ ID NO: 256 - DNA, 35 bp, Artificial Sequence
  Primer
tcaagtcagagtgagtgtgaccacaacatttcttt

SEQ ID NO: 257 - DNA, 35 bp, Artificial Sequence
  Primer
accagcgccaatgggacggtggaggttttatgaag

SEQ ID NO: 258 - DNA, 35 bp, Artificial Sequence
  Primer
cttcgtatggcttcgtatgccatgtcagtctgctt

SEQ ID NO: 259 - DNA, 35 bp, Artificial Sequence
  Primer
ggccggaaaaaaaaatgctcccctttaacaaccag

SEQ ID NO: 260 - DNA, 35 bp, Artificial Sequence
  Primer
gttctgagcagaaacacaccgttagggaagatggg

SEQ ID NO: 261 - DNA, 35 bp, Artificial Sequence
  Primer
tttgacctaatcatggaggccatggaagtgacagc

SEQ ID NO: 262 - DNA, 35 bp, Artificial Sequence
  Primer
gttttcctctctcctggccttgtctcagacccttg

SEQ ID NO: 263 - DNA, 35 bp, Artificial Sequence
  Primer
cataagaatgtaattaagtttaaacggggttgtga

SEQ ID NO: 264 - DNA, 35 bp, Artificial Sequence
  Primer
aattccaagatcaagatgccatcagttcagtttct

SEQ ID NO: 265 - DNA, 35 bp, Artificial Sequence
  Primer
gcttttggcagcatataaaatcatcttgagaaaga

SEQ ID NO: 266 - DNA, 35 bp, Artificial Sequence
  Primer
gctcacaatctctccatccttgaatagaacatgat

SEQ ID NO: 267 - DNA, 35 bp, Artificial Sequence
  Primer
tgactaaacaacaatgacaataacaaagagtctta

SEQ ID NO: 268 - DNA, 35 bp, Artificial Sequence
  Primer
caactccagccctgatttcccagaaccgtgcagga

SEQ ID NO: 269 - DNA, 35 bp, Artificial Sequence
  Primer
tgaatagatgaaacaaagcaaaacaaaggctctaa

SEQ ID NO: 270 - DNA, 35 bp, Artificial Sequence
  Primer
ctgccacatgaagcaaagatgggtctccagaattc

SEQ ID NO: 271 - DNA, 35 bp, Artificial Sequence
  Primer
caccttctctgaaaatagcatttttcaggtcaagg

SEQ ID NO: 272 - DNA, 35 bp, Artificial Sequence
  Primer
aggtccgtaagtcgctatctacaaaatggtcaact

SEQ ID NO: 273 - DNA, 35 bp, Artificial Sequence
  Primer
gcggagactgaggctcaacaagggggcttcagcaa

SEQ ID NO: 274 - DNA, 35 bp, Artificial Sequence
  Primer
tccaaggagctcatttcatcttggtcagagagacc

SEQ ID NO: 275 - DNA, 35 bp, Artificial Sequence
  Primer
aggactgggctgtgggtcctgagaccttctagact

SEQ ID NO: 276 - DNA, 35 bp, Artificial Sequence
  Primer
ggctaaagaggaatgtggggagcaaggctgggtca

SEQ ID NO: 277 - DNA, 35 bp, Artificial Sequence
  Primer
acttaaaaacagcttagttccactgtgatctatac

SEQ ID NO: 278 - DNA, 35 bp, Artificial Sequence
  Primer
ccactcagtacacacacagatctggaaacttcatc

SEQ ID NO: 279 - DNA, 35 bp, Artificial Sequence
  Primer
ttgtctctctgcctatatccttaggatctagaaaa

SEQ ID NO: 280 - DNA, 35 bp, Artificial Sequence
  Primer
aacttgtgacttctgtcatcatttctacgatctcc

SEQ ID NO: 281 - DNA, 35 bp, Artificial Sequence
  Primer
acactggtaatgtgttgacattaggacattgagtt

SEQ ID NO: 282 - DNA, 35 bp, Artificial Sequence
  Primer
aatcatggttttgccacttgttagctgtattacct

SEQ ID NO: 283 - DNA, 35 bp, Artificial Sequence
  Primer
gagatctagaacctcctgggaaagaaaagggttga

SEQ ID NO: 284 - DNA, 35 bp, Artificial Sequence
  Primer
gaatttggcaacttcgcctactttgacccgcagag

SEQ ID NO: 285 - DNA, 35 bp, Artificial Sequence
  Primer
tgtcctttttcattttgtaaaatttccagctcaga

SEQ ID NO: 286 - DNA, 35 bp, Artificial Sequence
  Primer
cttacaagttcagcagcttcactctttcaatcgag

SEQ ID NO: 287 - DNA, 35 bp, Artificial Sequence
  Primer
gactctcattaaattgagacctgaaataaattttc

SEQ ID NO: 288 - DNA, 35 bp, Artificial Sequence
  Primer
cagcaagaggtgaagtgataggtgagaagttattt

SEQ ID NO: 289 - DNA, 35 bp, Artificial Sequence
  Primer
gaaatagcaatattgaacctttagttgtggtttgt

SEQ ID NO: 290 - DNA, 35 bp, Artificial Sequence
  Primer
ccttcagtaaacaagtttgcagatccttcatcagg

SEQ ID NO: 291 - DNA, 35 bp, Artificial Sequence
  Primer
tgtatactttttgtcatttaaaggctgtgaacttg

SEQ ID NO: 292 - DNA, 35 bp, Artificial Sequence
  Primer
attggtatcacaaagactgaaggtcagcatatcta

SEQ ID NO: 293 - DNA, 35 bp, Artificial Sequence
  Primer
cagtcatgaggatgacacctagcatttgattgtaa

SEQ ID NO: 294 - DNA, 35 bp, Artificial Sequence
  Primer
cacagccactaaaataggatgagaatgatgtgagg

SEQ ID NO: 295 - DNA, 35 bp, Artificial Sequence
  Primer
gagagtttcatcctgatttaactaaggagatcata

SEQ ID NO: 296 - DNA, 35 bp, Artificial Sequence
  Primer
ccagcatcagggtcttttctgataagttgtctctt

SEQ ID NO: 297 - DNA, 35 bp, Artificial Sequence
  Primer
gagctcctcctgattccagacttggaagagcggct

SEQ ID NO: 298 - DNA, 35 bp, Artificial Sequence
  Primer
gtcccccctaacccttcttaccgtctctcctggta

SEQ ID NO: 299 - DNA, 35 bp, Artificial Sequence
  Primer
tcttggtcacatttgtcatccatggtgagaggtca

SEQ ID NO: 300 - DNA, 35 bp, Artificial Sequence
  Primer
tctgacccaatgcagctgatagagactacagagtt

SEQ ID NO: 301 - DNA, 20 bp, Artificial Sequence
  Primer
acacgacgctcttccgatct

SEQ ID NO: 302 - DNA, 20 bp, Artificial Sequence
  Primer
ctgaacccttgtcgccattc

SEQ ID NO: 303 - DNA, 70 bp, Artificial Sequence
  Primer
  misc_feature (30)..(37): n is a, c, g, or t
aatgatacggcgaccaccgagatctacacnnnnnnnnacactctttccctacacgacgctcttccgatct

SEQ ID NO: 304 - DNA, 69 bp, Artificial Sequence
  Primer
  misc_feature (25)..(32): n is a, c, g, or t
caagcagaagacggcatacgagatnnnnnnnncggtctcggcattcctgctgaacccttgtcgccattc